SEQUENCE LISTING (1 sequence)

SEQ ID NO: 1 - DNA, 5472 bp, Homo sapiens
  misc_feature (1079)..(1079): n is g or t
ataaaaaattaatgtaaagaaatatagctaaaagccactaggagaattaaaaccataagctaaaaaatgtttacttgacacataagaaagtagcaaaggaggaacagaaacaaaaagatacgagacaaattgaaaacgtatagcaaaatggtagaccaaaatccaaccattataagtgaagaaatgacacgacctgagtcacattagcaggactgctgagcactgtggggagaacagacatgggcaggaggtgagggacagtgttagtgccacaattcaggagtgacagggtggcggggactaaaggggaaagagggtgtgagggatgagaggggcagagagaagggctggagaagcaggaggtgaggaaaaggagcagaggaaagaattctaaagcagtagaagagcctggcagggggttctttgcattcggtatttaatacattttgtgtgactgccttaaaactaatgggctccttatgatttttttttaaaaaggggttacaaaaatatcaagtgtccaaataaaatatgcacactgcttagatgtgcatagttcacgaaaacgggcagtgctggagcgctggtgaagagcattgggactgcatggagccctcgcaactttgaggtgatgactacaggctcccggttgcaatagacagtaacaaaccctgcttctttgtattcaggagatgttctggactcacacagggaaactctggctagagaatgaggataactttaaatgcaacaacccagagtcacagatccatagtctgcgaaagtaaaacaggagctttgagaatttaattgtaatgcagttttgacacaggtctttcacagattggaattctaatcattcagggattaccaatattgtgctacctactgtatcaataaacaaaaaggaaactggtctctatgagaatctctacctggtgctttcagacaaaacttcaccaggtttaaagagaaaactcctgactctacacgtccattcccagggcgagctcactgtctggcatcaagttccccatggtgagtttccctgtacaagagtccaaggggagaggtaagtntcctttattttgctggatgtagtttaatattacctgaggtgaggtaaggtaaggcaaagggtgggaggcagggagtccagttcagggacggggattccaggaggagaagtgaaggggaaggggctgggcgcagccttggggtctctccctggtttccacagacagatccttgtccaggactcaggcacacagtgtgacaaagatgcttggtgtaggagaagaggnatcaggacgaagtcccaggtcccgggcggggctctcagggtctcaggctccaagggccgtgtctgcantggggaggcgccgcgttggggattctccactcccctgagtttcacttctnccaacctgcgtcgggtccttcttcctgaatactcatgacgcgtccccaattcccactcccattgggtgtcgggttctagagaagccaatcagcgtctccgcagtcccggttctaaagtccccagtcacccacccggactcacattctccccagaggccgagatgcgggtcatggcgccccgagccctcctcctgctgctctcgggaggcctggccctgaccgagacctgggcctgtgagtgcggggttgggagggaagcggcctctgcggagaggagcgaggggcccgcccggcgagggcgcaggacccggggagccgcgcagggaggtgggtcgggcgggtctcagcccctcctcgcccccaggctcccactccatgaggtatttcgacaccnccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaactacaagcgccaggcacagnctgaccgagtgancctgcggaacctgcgcggctactacaaccagagcgaggacggtgagtgaccccggcccggggcgcaggtcacgacccctccccatcccccacggacggcccgggtcgccccgagtctccccgtctgagatccaccccaaggtggatctgcggaacccgcccagaccctcgaccggagagagccccagtcgcctttacccggtttcattttcggtttaggccaaaatccccgcgggttggtcggggcggggcggggctcgggggactgggctgaccgcgggggcggggccagggtctcacaccctccagaggatgtatggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcaggtaccaggggcagtggggagccttccccatctcctatagatctcccgggatggcctcccacgaggaggggaggaaaatgggatcagcactggaatatcgccctcccttgaatggagaatggcatgagttttcctgagtttcctctgagggccccctctgctctctaggacaattaagggatgaagtctctgaggaaatggaggggaagacagtccctggaatactgatcaggggtctcctttgaccactttgaccactgcagcagctgtggtcaggctgctgacctttctctcaggccttgttctctgcctcacactcaatgtgtctgaaggtttgattccagcttttctgagtcctgcagcctccactcaggtcaggaccagaagtcgctgttcctccctcagagactagaactttccaatgaataggagattatcccaggtgcctgtgtccaggctggcgtctgggttctntgccgccttccccaccccaggtgtcctgtccnttctcaggatggtcacatgggcgctgctggagtgtcccaagagagatgcaaagtgtctgaattttctgactcttcccgtcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcangagcccctcaccctgagctggggtaaggaggggaatggggggtcacatctcttatcagagaaagcagaagtccttctggagcccttcagccgggtcagggctgaggcttgggggtcagggcccctcaccttctcctcctttcccagagccatcttcccagcccaccatccccatcatgggcatcgttgctggcctggctgtcctggttgtcctagctgtccttggagctgtggtcaccgctatgatgtgtaggaggaagagctcaggtagggaaggggtgaagagcggggtctgggttttcttgtcccactgggagtttcaagccccaggtagaagtgtgccccgccttgttactggaagcaccatccacacatgggccatcccagcctgggaccctgtgtgccagcacttactcttttgtgaagcacatgtgacaatgaaggacggatgtatcaccttgatgattatggtgttggggtcctgattccagcattcatgagtcaggggaaggtccctgctaaggacagaccttaggagggcagttggtccagaacccacaactgctttccccatgtttcctgatcctgccctgggtctgcagtcgtagttctggaaacttctcttgggtccaagactaggaggttcccctaagatcacatggccctgcctcctcccagtcccctcatagggcattttcttcccacaggtggaaaaggagggagctgctctcaggctgcgtgtaagtgatggcggcgggcgtgtggaggagctcacctactccataattcctcttgtcccacatctcctgcgggctctgaccaggtctttttttttgttctaccccaggcagcaacagtgcccagggctctgatgagtctctcatcacttgtaaagcctgaattctggggagctgaagtggtcgggggtggggcagagggaaaaggcctgggtaatggggattctttgattgggacgtttcgagtgtgtggtgggccgttcagagtgtcatcacttaccatgactgacctgaatttgttcatgactattgtgttctgtagcctgagacagctgcctgtgtgggactgagatgcaggatttcttcacacctctcctttgtgacttcaagagcctctggcatctctttctgcaaaggcgtctgaatgtgtctgcgttcctgttagcataatgtgaggaggtggagagacagcccacccccgtgtccaccgtgacccctgtccccacactgacctgtgttccctccccgatcatctttcctgttccagagaggtggggctggatgtctccatctctgtctcaaattcatggtgcactgagctgcaacttcttacttccctaatgaagttaagaacctgaatataaatttgtgttctcaaatatttgctatgaagcgttgatggattaattaaataagtcaattcctagaagttgagagagcaaataaagacctgagaaccttccagaatttgcatgttcgctgtgctgagtctgttgcaggtgggggtggggaaggctgtgaggagccgagtgtggacggggcctgtgcctagttgctgttcagttcttcatgggctttatgtggtcagtcctcagctgggtcaccttcactgctccattgtccttgtcccttcagtggaaacttgtccagcggaagctgtgaccacagaggctcacccatcgcccagggcagcccctgcacacgggagtccctgtgctttctgagacaaattttcagacccattcagctcctgccctccttctagggctcctcttctgctttggtctcctgccctctctcccttccctgattccagtgatcttcgtgctgactccaatcccaactcatgaatctaaagcagagcctaatttagatttgtatttgtttgtaaaattgggtccatagtctagaattgttccttcctgaagagagaaacctgatcgtgtgctgcagtgtgcggggcggttggtgtgggaggagggataggggagggaggacacacaagcagccctgctgagaaaagtacaggcggc

(19) United States
(12) Patent Application Publication
(10) Pub. No.: US 2006/0183146 A1
(43) Pub. Date: Aug. 17, 2006

(54) Genetic markers in the HLA-C gene associated with an adverse hematological response to drugs

(76) Inventors: Maria Athanasiou, Derby, CT (US); Stanton Gerson, Hunting Valley, OH (US)

Correspondence Address: GENAISSANCE PHARMACEUTICALS, 5 SCIENCE PARK, NEW HAVEN, CT, 06511, US

(21) Appl. No.: 11/351,601

(22) Filed: Feb. 10, 2006

Related U.S. Application Data
(60) Provisional application No. 60/652,135, filed on Feb. 11, 2005.

(51) Int. Cl. C12Q 1/68

(52) U.S. Cl. 435/6

(57) ABSTRACT

Genetic markers in the HLA-C gene associated with adverse hematological response to drug therapy are disclosed. Compositions and methods for detecting and using these HLA-C markers in a variety of clinical applications are disclosed. Such applications include methods for testing an individual for susceptibility for an adverse hematological response, methods of selecting the appropriate drug therapy for patients based on the presence or absence of a HLA-C marker, and products comprising a drug with hematological toxicity that are approved for treating patients lacking a genetic marker.

### CROSS REFERENCE TO RELATED APPLICATIONS

This application claims the benefit of U.S. Application No. 60/652,135, filed Feb. 11, 2005.

### FIELD OF THE INVENTION

This invention relates to the field of pharmacogenetics. More specifically, this invention relates to certain variants of the gene encoding major histocompatibility complex, class I, subclass alpha, C (HLA-C) that are associated with an adverse hematological response to drugs.

### BACKGROUND OF THE INVENTION

Adverse hematological events induced by drug therapy are a serious health risk and can be fatal. In the United States, the labels of over 40 currently marketed prescription drugs include a warning of a risk for patients treated with the drug to develop neutropenia and or agranulocytosis (Physician's Desk Reference (59th ed., 2005, hereinafter “PDR”), with antithyroid medications and sulfonamides being the most common drugs associated with agranulocytosis (Berliner N., et al. Hematology 2004, p. 63-79). Neutropenia is typically defined as the presence of an abnormally small number of neutrophils in the circulating blood (Stedman's Medical Dictionary 1207 (26th ed. 1995). Neutrophils, which constitute 50-75% of the total circulating leukocytes, are granulocytes that play a key role in inflammatory and immune responses to invading infectious agents and tumor cells (Barreda, D. R. et al. (2004) Developmental and Comparative Immunology 28: 509-554). Agranulocytosis, an acute neutropenic condition in which the absolute neutrophil count (ANC) is typically less than 500/mm³ blood (Stedman's Medical Dictionary 39 (26th ed. 1995)), is an adverse event reported with numerous drugs. The risk of this adverse hematological response is highlighted on the labels for five currently marketed drugs in a “black box” warning, which is the strongest safety warning the United States Food and Drug Administration (FDA) may impose before banning marketing of a drug (Ostruousky, O. et al. (2003) Tissue Antigens 62: 483-491; PDR).

One drug with a black box warning for agranulocytosis is clozapine, a tricyclic dibenzodiazepine derivative marketed by several companies; with perhaps the best known clozapine drug product being CLOZARIL® (clozapine) tablets marketed by Novartis. Clozapine, which is classified as an “atypical” antipsychotic drug based on its dopamine receptor binding profile and effects on various dopamine mediated behaviors (PDR, p. 2280), has demonstrated superior efficacy over chlorpromazine for treatment-resistant schizophrenia and is relatively free of the extrapyramidal side effects such as parkinsonism, tardive dyskinesia, and dystonis associated with chlorpromazine and other classical antipsychotics such as thioridazine, fluphenazine, haloperidol, flupenthixol, molindone, loxapine, and pimozide (Dettling M. et al. (2001) Pharmacogenetics 11:135-141; Ostrousky et al., supra; Theodoropoulou, St. et al. (1997) Neuropsychobiology 36:5-7; Lahdelma, L. et al. (2001) 21:4-7). Clozapine may also have clinical utility in treating other disoders, including psychosis secondary dopaminergic therapy or coexisting psychiatric disorders in Parkinson's disease, other psychotic disorders, affective disorders, personality disorders, dyskinesias and related disorders, dementia, mental retardation and polydipsia/hyponatramia.

However, because of the significant risk for agranulocytosis (an estimated cumulative incidence of about 1.3% at 1 year of clozapine therapy) (PDR, p. 2281), clozapine is approved only for “the management of severely ill schizophrenia patients who fail to respond adequately to standard drug treatment for schizophrenia” (Id.) and is available only through a distribution system that ensures monitoring of white blood ell (WBC) counts according to a complicated algorithm prior to delivery of the next supply of medication (supra, p. 2280-2282.). This restricted distribution is accomplished via patient registries managed by the maufacturers of clozapine drug products (i.e., Novartis' Clozaril Patient Registry and Mylan's Clozapine Presription Access System). The prescribing physician must provide weekly reporting of white blood cell counts (WBC) and absolute neutrophil counts (ANC) for the first six months of treatment, and at least bi-weekly thereafter (supra). This blood testing schedule is based on the observations that the majority of CIA cases occur within the first 18 weeks of treatment, that a significant number still occur in the first 6 months of treatment and that the risk declines significantly after 6 months, but never goes to zero (Theodoropoulou et al., supra). The initial “threshold” for WBC and ANC is 3000/mm³ and 1500/mm³, respectively, meaning that should either of these numbers be reached, treatment must be interrupted, but may be resumed (supra). If, however, a patient's WBC falls below 2000 mm³, or ANC falls below 1000/mm³, treatment must be permanently discontinued (supra). There is also a short period of monitoring that must occur at the end of the treatment period (supra). Because of this unique distribution system, not to mention the underlying risk of agranulocytosis, utilization of clozapine is limited. Compliance with the blood monitoring system is particularly difficult in the schizophrenia patient population and psychiatrists are hesitant to prescribe the medication, even for treatment-resistant patients.

Because of the proven clinical benefits of clozapine, there has been much research into understanding the pathogenic mechanisms of clozapine-induced agranulocytosis (CIA) with a goal of being able to identify patients who are at risk for CIA and agranulocytosis induced by other drugs (Claas, F. H. J, (1989) Psychopharmacology 99:S113-S117). This research has produced substantial evidence that there is a genetic basis to CIA. For example, associations of certain human leukocyte-antigen (HLA)-haplotypes with CIA in Jewish and non-Jewish Caucasian pateints have been reported (Dettling, M. et al. aupra; Dettling M. et al. (2001) Arch. Gen Psychiatry 58:93-94; Amar, A. et al., (1998) Int. J. Neuropsychopharmacol 1:41-44; Yunis, J. J. et al. (1995) Blood 86:1177-1183; Lieberman et al. (1990) Arch Gen Psychiatry 47:945-948). These CIA-associated haplotypes include three alleles of the HLA-DQB1 gene: DQB1*0201 and DQB*0302 alleles in Jewish patients and DQB1*0502 in non-Jewish Caucasian patients. However, two other studies failed to show an association between any specific HLA haplotype and CIA (Theodoropoulou et al., supra; Class et al. (1992) Drug Safety 7(suppl1):3-6).

Based on these contradictory reports about the role of HLA genetic variation in CIA, it would be useful to examine genes in the HLA complex to see if genetic variability in any of these genes is involved in susceptibility for drug-induced agranulocytosis, and in particular CIA. Such an understanding could lead to a genetic test that would identify a population of patients at reduced risk of developing an adverse hematological response. The development and commercialization of such a test has the potential to improve the safety of currently marketed drugs known to induce neutropenia, granulocytopenia and agranulocytosis, and in the case of clozapine, safely increase the use of a highly efficacious drug.

Class I antigens in the major histocompatibiltiy complex are expressed on all somatic cells of an adult individual, but are expressed in a particularly high concentration on lymphocytes. Class I proteins are heterodimers consisting of a cell surface glycoprotein noncovalently associated with a beta-microglobulin (Cianetti, L. et al. (1989) Immuniogenetics 29:80-91). The gene encoding HLA-C (FIG. 1), which is one of three class I HLA genes (HLA-A and HLA-B) within the 6p21.3 locus, encodes a protein of 366 amino acids. The HLA-C gene contains seven exons: exon 1 encodes the leader peptide, exons 2, 3, and 4 encode the extracellular domains, and exons 5, 6, and 7 encode the transmembrane and cytoplasmic portions (Mallisen, M. et al. (1982) Proc. Natl. Acad. Sci USA 79(3):893-7).

### SUMMARY OF THE INVENTION

Accordingly, the inventors herein have discovered markers in the HLA-C gene that are associated with adverse hematological response to a drug. These HLA-C markers have a variety of pharmacogenetic research and clinical applications.

In a first aspect, the invention provides a method for testing an individual for susceptibility for an adverse hematological response to treatment with a drug comprising detecting the presence or absence in the individual of a HLA-C marker, and generating a test report for the individual, wherein if the HLA-C marker is present, then the test report indicates that the individual is susceptible for the adverse hematological response, and if the HLA-C marker is not present, then the test report indicates that the individual is not susceptible for the hematological adverse response.

In another aspect, the invention provides a method of testing an individual for the presence or absence of a genetic marker that is associated with an adverse hematological response to treatment with a drug comprising determining the copy number of a polymorphism in the HLA-C gene that is associated with the adverse hematological adverse response, using the determined copy number to assign to the individual the presence of absence of the marker, and generating a test report which indicates whether the marker is present or absent in the individual.

In yet another aspect, the invention provides a method of predicting whether an individual is susceptible for a hematological adverse response to treatment with a drug comprising determining the presence or absence in the individual of a HLA-C marker, and making a prediction based on the results, wherein if the HLA-C marker is present, then the prediction is that the individual is likely to exhibit the hematological adverse response if treated with the drug and if the HLA-C marker is absent, the prediction is that the individual is not likely to exhibit the hematological adverse response.

In another aspect, the invention provides a kit for detecting a HLA-C marker comprising a set of one or more oligonucleotides designed for identifying each of the alleles at each polymorphic site in the HLA-C marker.

In another aspect, the invention provides a method of selecting a suitable therapy for an individual who is a candidate for treatment with a drug that has a propensity for inducing an adverse hematological response, comprising determining the presence or absence in the individual of a HLA-C marker, and selecting the therapy based on the results.

In another aspect, the invention provides a method for seeking regulatory approval for a new indication for a pharmaceutical formulation comprising a drug known to have a propensity to induce an adverse hematological response.

In another aspect, the invention provides a method of advertising a drug product which comprises a drug that has a propensity to induce an adverse hematological response, the method comprising promoting to a target audience the use of the drug product in individuals who test negative for a HLA-C marker.

In another aspect, the invention provides a manufactured drug product comprising a drug with a propensity to induce an adverse hematological response and prescribing information which states that the drug product is indicated for patients who test negative for a HLA-C marker. The invention also provides a method manufacturing such a pharmacogenetic drug product.

### BRIEF DESCRIPTION OF THE FIGURES

FIG. 1A-1C illustrates a reference sequence for the HLA-C gene (contiguous lines; SEQ ID NO:1), with the start and stop positions of each region of coding sequence indicated with a bracket ([ or ]) and the numerical position below the sequence and the polymorphic site(s) and polymorphism(s) indicated by the variant nucleotide positioned below the polymorphic site in the sequence.

### DETAILED DESCRIPTION OF THE INVENTION

I. Definitions

So that the invention may be more readily understood, certain terms are first defined.

As used in the specification and the claims, “a” or “an” means one or more unless explicitly stated otherwise. As used herein, “another” means at least a second or more.

“Adverse hematological response” means any one or more of the following conditions that is exhibited by a subject following treatment with a drug: neutropenia (and its various synonyms such as neutrophilic leukopenia, neurtrophilopenia), granulocytopenia (and its various synonyms such as granulopenia, hypogranulocytosis), and agranulocytosis. Preferably, an adverse hematological response is a drug toxicity criteria established by any medical or scientific authority. For example, the National Cancer Institute classifies the toxicity of drugs with respect to neutrophil and granulocyte levels into 4 grades of increasing toxicity: Grade 1=*1.5−<2.0×109/L or *1500−<2000/mm³; Grade 2=*1.0−<1.5×109/L or *1000−<1500/mm³; Grade 3=*0.5−<1.0×109/L or *500−<1000/mm³; and Grade 4=<0.5×109/L or <500/mm³. In more preferred embodiments, the adverse hematological response is a neutrophil/granulocyte count within Grade 3 or Grade 4. In a particularly preferred embodiment, the adverse hematological response is a neutrophil/granulocyte count classified as Grade 4.

“Allele” is a particular form of a gene or other genetic locus, distinguished from other forms by its particular nucleotide sequence, the term allele also includes one of the alternative polymorphisms (e.g., a SNP) found at a polymorphic site. In some contexts, it will be readily apparent to the skilled artisan that the term allele refers to the form of a locus that is present on a single chromosome in a somatic cell obtained from an individual; if the locus is on an autosomal chromosome, then the somatic cell in the individual will normally have two alleles for the locus. If these alleles have identical sequences, the individual is homozygous for that locus, and if the two alleles have different sequences, then the individual is heterozygous for the locus. If the locus is on a sex chromosome, then somatic cells from female individuals normally have two alleles, which may have the same or different sequences, while somatic cells from male individuals normally only has one allele for the locus.

“Disease” refers to an interruption, cessation, or disorder of one or more body functions, structures, systems or organs.

“Drug” includes any therapeutic or prophylactic compound, substance or agent including, without limitation, a small molecule, protein, vaccine, antibody or nucleic acid, that (a) is known to induce an adverse hematological response in some measurable percentage of individuals exposed to the drug or (b) is being tested for a propensity to induce an adverse hematological response using one of the methods of the invention. In the description herein of some embodiments of the invention, it will be evident to the skilled artisan that the term drug can include a pharmaceutical composition or drug product comprising a therapeutic or prophylactic compound, substance or agent.

“Gene” is a segment of DNA that contains the coding sequence for a protein, wherein the segment may include promoters, exons, introns, and other untranslated regions that control expression.

“HLA-C Marker” in the context of the present invention is a specific copy number of a specific polymorphism that is associated with an adverse hematological response. Preferred HLA-C markers are those shown in Table A-1 (Appendix A), as well as genetic markers that are highly correlated with any marker in Table A-1 and/or are replaced by the same copy number of a substitute polymorphism, each of which is referred to herein as an alternate genetic marker. A substitute polymorphism comprises a sequence that is similar to that of any of the markers shown in Table A-1 (Appendix A), but in which the allele at one or more of the specifically identified polymorphic sites in that marker has been substituted with the allele at a different polymorphic site, whose substituting allele is in high linkage disequilibrium (LD) with the allele at the specifically identified polymorphic site. A linked polymorphism is any type of polymorphism, including a haplotype, which is in high LD with any one of the markers shown in Table A-1 (Appendix A). Two particular alleles at different loci on the same chromosome are said to be in LD if the presence of one of the alleles at one locus tends to predict the presence of the other allele at the other locus. Alternate genetic markers, which are further described below, may comprise types of variations other than SNPs, such as indels, RFLPs, repeats, etc.

“Genotype” is an unphased 5′ to 3′ sequence of the two alleles, typically a nucleotide pair, found at a set of one or more polymorphic sites in a locus on a pair of homologous chromosomes in an individual.

“Genotyping” is a process for determining a genotype of an individual.

“Granulocytopenia” is a condition in which a subject has less than the normal number of granular leukocytes in the blood, typically, granulocytopenia refers to a granulocyte count of less than 1500/mm³.

“Haplotype pair” refers to the two haplotypes found for a locus in a single individual.

“Haplotyping” refers to any process for determining one or more haplotypes in an individual, including the haplotype pair for a particular set of PS, and includes use of family pedigrees, molecular techniques and/or statistical inference.

“Isolated” is typically used to reflect the purification status of a biological molecule such as RNA, DNA, oligonucleotide, or protein, and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with the methods of the present invention.

“Locus” refers to a location on a chromosome or DNA molecule corresponding to a gene, a physical feature such as a polymorphic site, or a location associated with a phenotypic feature.

“Normal” as used herein in connection with the quantity in a subject of any clinical parameter (such as any type of blood cell or one of its hematopoietic precursors) means a specific number or numerical range of that parameter that is typically observed in healthy subjects of similar age, weight, and or gender, or that would be understood by a clinical to be normal. Conversely, “abnormal” refers to a specific number or numerical range for a clinical parameter that is lower or higher than a normal number or normal numerical range, or that would be understood by a clinical to be abnormal.

“Nucleotide pair” is the set of two nucleotides (which may be the same or different) found at a polymorphic site on the two copies of a chromosome from an individual.

“Oligonucleotide” refers to a nucleic acid that is usually between 5 and 100 contiguous bases in length, and most frequently between 10-50, 10-40, 10-30, 10-25, 10-20, 15-50, 15-40, 15-30, 15-25, 15-20, 20-50, 20-40, 20-30 or 20-25 contiguous bases in length. The sequence of an oligonucleotide can be designed to specifically hybridize to any of the allelic forms of a locus; such oligonucleotides are referred to as allele-specific probes. If the locus is a PS comprising a SNP, the complementary allele for that SNP can occur at any position within an allele-specific probe. Other oligonucleotides useful in practicing the invention specifically hybridize to a target region adjacent to a PS with their 3′ terminus located one to less than or equal to about 10 nucleotides from the PS, preferably ≦ about 5 nucleotides. Such oligonucleotides hybridizing adjacent to a PS are useful in polymerase-mediated primer extension methods and are referred to herein as “primer-extension oligonucleotides.” In a preferred embodiment, the 3′-terminus of a primer-extension oligonucleotide is a deoxynucleotide complementary to the nucleotide located immediately adjacent to the PS.

“Phased sequence” refers to the combination of nucleotides present on a single chromosome at a set of polymorphic sites, in contrast to an unphased sequence, which is typically used to refer to the sequence of nucleotide pairs found at the same set of PS in both chromosomes.

“Polymorphic site” or “PS” refers to the position in a genetic locus or gene at which a SNP or other nonhaplotype polymorphism occurs. A PS is usually preceded by and followed by highly conserved sequences in the population of interest and thus the location of a PS is typically made in reference to a consensus nucleic acid sequence of thirty to sixty nucleotides that bracket the PS, which in the case of a SNP polymorphism is sometimes referred to as a context sequence for the SNP. The location of the PS may also be identified by its location in a consensus or reference sequence relative to the initiation codon (ATG) for protein translation. The skilled artisan understands that the location of a particular PS may not occur at precisely the same position in a reference or context sequence in each individual in a population of interest due to the presence of one or more insertions or deletions in that individual as compared to the consensus or reference sequence. Moreover, it is routine for the skilled artisan to design robust, specific and accurate assays for detecting the alternative alleles at a polymorphic site in any given individual, when the skilled artisan is provided with the identity of the alternative alleles at the PS to be detected and one or both of a reference sequence or context sequence in which the PS occurs. Thus, the skilled artisan will understand that specifying the location of any PS described herein by reference to a particular position in a reference or context sequence (or with respect to an initiation codon in such a sequence) is merely for convenience and that any specifically enumerated nucleotide position literally includes whatever nucleotide position the same PS is actually located at in the same locus in any individual being tested for the presence or absence of a genetic marker of the invention using any of the genotyping methods described herein or other genotyping methods well-known in the art.

“Polymorphism” refers to one of two or more genetically determined alternative sequences or alleles that occur for a gene or a genetic locus in a population. As used herein, the term polymorphism includes, but is not limited to (a) a sequence of as few as one nucleotide that occurs at a polymorphic site (as defined above), which is also referred to herein as a single nucleotide polymorphism (SNP) and (b) a sequence of nucleotides that occur on a single chromosome at a set of two or more polymorphic sites in the gene or genetic locus of interest, which is also referred to herein as a haplotype. The different alleles of a polymorphism typically occur in a population at different frequencies, with the allele occurring most frequently in a selected population sometimes referenced as the “major” or “wildtype” allele. Diploid organisms may be homozygous or heterozygous for the different alleles that exist. A biallelic polymorphism has two alleles, and the minor allele may occur at any frequency greater than zero and less than 50% in a selected population, including frequencies of between 1% and 2%, 2% and 10%, 10% and 20%, 20% and 30%, etc. A triallelic polymorphism has three alleles. In addition to SNPs and haplotypes, examples of polymorphisms include restriction fragment length polymorphisms (RFLPs), variable number of tandem repeats (VNTRs), dinucleotide repeats, trinucleotide repeats, tetranucleotide repeats, simple sequence repeats, insertion elements such as Alu, and deletions of one or more nucleotides.

“Treat” or “Treating” means to administer a drug internally or externally to a patient having one or more disease symptoms for which the drug has known therapeutic activity. Typically, the drug is administered in an amount effective to alleviate one or more disease symptoms in the treated patient or population, whether by inducing the regression of or inhibiting the progression of such symptom(s) by any clinically measurable degree. The amount of a drug that is effective to alleviate any particular disease symptom (also referred to as the “therapeutically effective amount”) may vary according to factors such as the disease state, age, and weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every patient, it should alleviate the target disease symptom(s) in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

II. Composition and Phenotypic Effect of HLA-C Markers of Adverse Drug Response

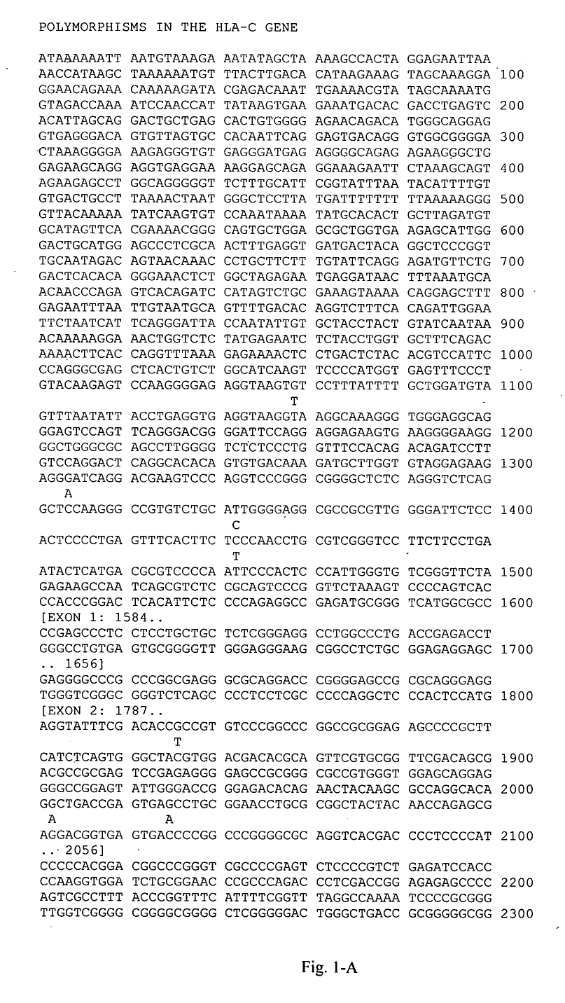

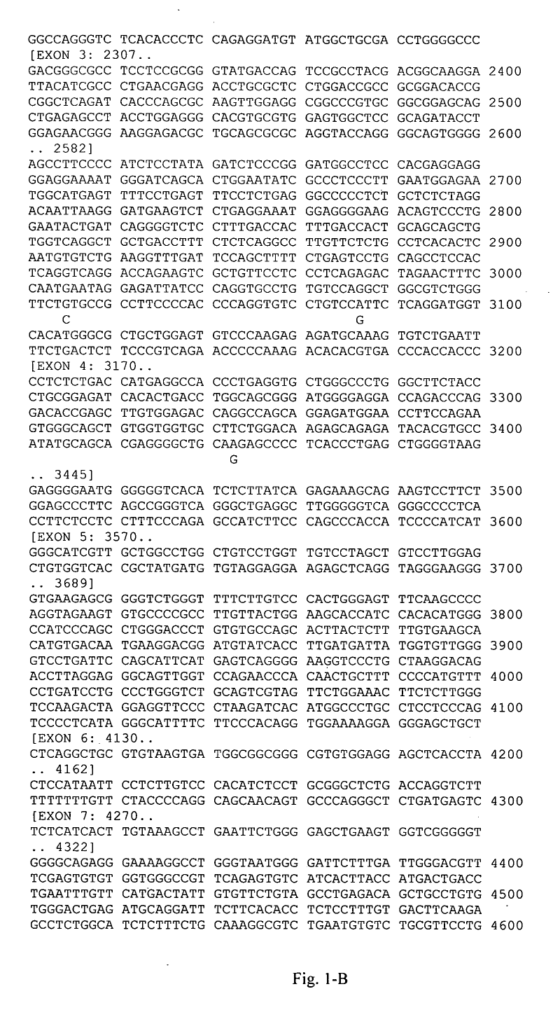

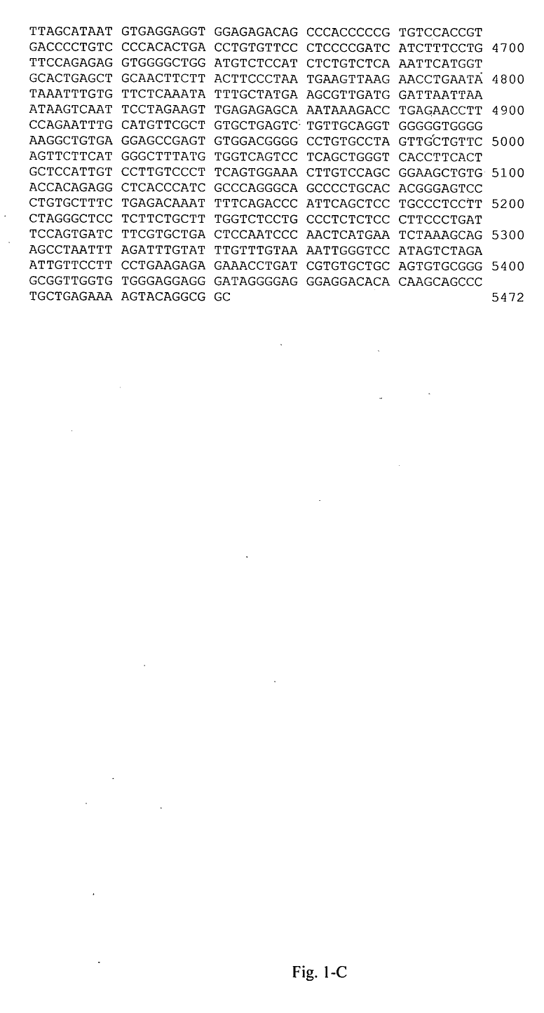

As described above and in the examples below, genetic markers according to the present invention are associated with an adverse hematological response to treatment with a drug, and are referred to herein as HLA-C markers. Each HLA-C marker of the invention is a combination of a particular polymorphism associated with the adverse hematological response and a copy number of that polymorphism. Preferably, the polymorphism is one of the markers shown in Table A-1 (Appendix A), each of which contains a sequence for a specific set of PS in the HLA-C gene. The locations of these marker PS in the HLA-C gene are at positions corresponding to those identified in FIG. 1/SEQ ID NO:1 (see Table A-2 in Appendix A for a summary of the PS location and the alternative nucleotide alleles that occur at each PS). In describing the PSs in the markers of the invention, reference is made to the sense strand of a gene for convenience. However, as recognized by the skilled artisan, nucleic acid molecules containing a particular gene may be complementary double stranded molecules and thus reference to a particular site on the sense strand refers as well to the corresponding site on the complementary antisense strand.

As described in more detail in the examples below, the genetic markers of the invention are based on the discovery by the inventors of associations between particular copy numbers of certain polymorphisms in the HLA-C gene and clozapine-induced agranuloctyosis. Individuals having the copy number indicated for each of the polymorphisms shown in Appendix A were more likely to develop agranulocytosis in response to clozapine treatment relative to individuals having other copy numbers of those polymorphisms. Moreover, as shown in Table 1 below, the association between the presence of these genetic markers and susceptibility for CIA is statistically significant across, respectively, all ethnicities and Caucasians only.

In addition, the skilled artisan will appreciate that all of the embodiments of the invention described herein may frequently be practiced using an alternate genetic marker for any of the genetic markers in Table A-1 (Appendix A). Alternate genetic markers are readily identified by determining the degree of linkage disequilibrium (LD) or the degree of correlation between an allele at a PS in Table A-2 (Appendix A) and a candidate substituting allele at a polymorphic site located elsewhere in the HLA-C gene or on chromosome 6. Similarly, alternate genetic markers comprising a linked polymorphism are readily identified by determining the degree of LD between a marker in Table A-1 (Appendix A) and a candidate linked polymorphism located elsewhere in the HLA-C gene or on chromosome 6. The candidate substituting allele or linked polymorphism may be a polymorphism that is currently known. Other candidate substituting alleles and linked polymorphisms may be readily identified by the skilled artisan using any technique well-known in the art for discovering polymorphisms.

The degree of LD between a genetic marker in Table A-1 (Appendix A) and a candidate alternate polymorphism may be determined using any LD measurement known in the art. LD patterns in genomic regions are readily determined empirically in appropriately chosen samples using various techniques known in the art for determining whether any two alleles (e.g., between SNPs at different PSs or between two haplotypes) are in linkage disequilibrium (GENETIC DATA ANALYSIS II, Weir, Sinauer Associates, Inc. Publishers, Sunderland, Mass., 1996). The skilled artisan may readily select which method of determining LD will be best suited for a particular sample size and genomic region.

One of the most frequently used measures of linkage disequilibrium is Δ², which is calculated using the formula described by Devlin et al. (Genomics 29(2):311-22 (1995)). Δ² is the measure of how well an allele X at a first locus predicts the occurrence of an allele Y at a second locus on the same chromosome. The measure only reaches 1.0 when the prediction is perfect (e.g., X if and only if Y).

In preferred alternate genetic markers, the locus of a substituting allele or a linked polymorphism is in a genomic region of about 100 kilobases spanning the HLA-C gene, and more preferably, the locus is in the HLA-C gene. Other preferred alternate genetic markers are those in which the LD or correlation between the relevant alleles (e.g., between the substituting SNP and the substituted SNP, or between the linked polymorphism and the haplotype) has a Δ² or r² (the square of correlation coefficient) value, as measured in a suitable reference population, of at least 0.75, more preferably at least 0.80, even more preferably at least 0.85 or at least 0.90, yet more preferably at least 0.95, and most preferably 1.0. The reference population used for this Δ² or r² measurement preferably reflects the genetic diversity of the population of patients to be treated with a drug associated with the adverse hematological response (such as clozapine). For example, the reference population may be the general population, a population using the drug, a population diagnosed with a particular condition for which the drug shows efficacy (such as schizophrenia in the case of CIA), or a population of similar ethnic background.

Preferred genetic markers of the invention comprise any of the markers in Table A-1 (Appendix A).

Individuals having any of the genetic markers described herein are susceptible to an adverse hematological response to clozapine and other drugs that induce this adverse response via one or more mechanisms in common. In some embodiments of the present invention, the adverse hematological response is due to the destruction of peripheral blood neutrophils (PMNs) and their hematopoietic precursors by cytotoxic antibodies generated against a neutrophil protein modified by the drug or a reactive metabolite thereof. In other embodiments, the drug induces the adverse response via suppression of hematopoiesis in the bone marrow. In still other embodiments, the drug binds to a neutrophil protein in a manner that induces apoptosis of neutrophils or a hematopoietic precursor. In some embodiments, a combination of these mechanisms underlying the etiology of the adverse hematological response associated with the genetic markers of the invention.

In preferred embodiments, the drug is an antithyroid medication or a sulfonamide. In other preferred embodiments, the approved label of the drug contains a precaution or a warning that the drug is associated with a risk for neutropenia or agranulocytosis. In more preferred embodiments, the drug is any of the following compounds or a pharmaceutically acceptable salt thereof: (1) clozapine; (2) quinapril; (3) moexipril; (4) benazepril; (5) enalapril; (6) perindopril erbumine; (7) carbamazepine; (9) lisinopril; (10) trandolapril; (11) ticlopidine; (12) captotril; (13) benazepril; (14) ramipril; (15) penicillamine; (16) propafenone; (17) sulfamethoxazole; (18) zonisamide; (19) leflunomide; (20) sulfacetamide; (21) prednisolone; (22) timolol; (23) dapsone; (24) ofloxacin; (25) levofloxacin; (26) sulfisoxazole; (27) promethazine; (28) amoxicillin; (29) mebendazole; (30) brinzolamide; (31) procainamide and (32) tocainide. In even more preferred embodiments, the drug is any of the following compounds or a pharmaceutically acceptable salt thereof: clozapine, carbamazepine, ticlopidine, procainamide or tocainide. In particularly preferred embodiments the drug is clozapine.

III. Detecting HLA-C Markers of Adverse Drug Response

In all of the embodiments of the invention, the skilled artisan will appreciate that detecting the presence or absence of a specific genetic marker in a marker group in an individual is also literally equivalent to detecting the presence or absence of the same copy number of a substitute, linked or correlated polymorphism for the polymorphism in that specific marker in which Δ²=1 for the linkage disequilibrium or the correlation coefficient=1 between the substituted polymorphism in that marker and the substituting polymorphism.

The presence in an individual of a genetic marker of the invention may be determined by any of a variety of methods well known in the art that permits the determination of whether the individual has the required copy number of the polymorphism comprising the marker. For example, if the required copy number is 1 or 2, then the method need only determine that the individual has at least one copy of the polymorphism. In preferred embodiments, the method provides a determination of the actual copy number.

Typically, these methods involve assaying a nucleic acid sample prepared from a biological sample obtained from the individual to determine the identity of a nucleotide or nucleotide pair present at one or more polymorphic sites in the marker. Nucleic acid samples may be prepared from virtually any biological sample. For example, convenient samples include whole blood, serum, semen, saliva, tears, fecal matter, urine, sweat, buccal matter, skin and hair. Preferred samples contain only somatic cells, and such samples would typically be required when the locus is on an autosomal or X chromosome. Nucleic acid samples may be prepared for analysis using any technique known to those skilled in the art. Preferably, such techniques result in the production of genomic DNA sufficiently pure for determining the genotype or haplotype pair for a desired set of polymorphic sites in the nucleic acid molecule. Such techniques may be found, for example, in Sambrook, et al., Molecular Cloning: A Laboratory Manual (Cold Spring Harbor Laboratory, New York) (2001), incorporated herein by reference.

For markers in which the specified polymorphism is a haplotype, the copy number of the haplotype in the nucleic acid sample may be determined by a direct haplotyping method or by an indirect haplotyping method, in which the haplotype pair for the set of polymorphic sites comprising the marker is inferred from the individual's haplotype genotype for that set of PSs. The way the nucleic acid sample is prepared depends on whether a direct or indirect haplotyping method is used.

Direct haplotyping methods typically involve treating a genomic DNA sample isolated from a blood or cheek sample obtained from the individual in a manner that produces a hemizygous DNA sample that contains only one of the individual's two alleles for the locus which, as readily understood by the skilled artisan, may be the same allele or different alleles, and detecting the nucleotide present at each PS of interest. The nucleic acid sample may be obtained using a variety of methods known in the art for preparing hemizygous DNA samples, which include: targeted in vivo cloning (TIVC) in yeast as described in WO 98/01573, U.S. Pat. No. 5,866,404, and U.S. Pat. No. 5,972,614; generating hemizygous DNA targets using an allele specific oligonucleotide in combination with primer extension and exonuclease degradation as described in U.S. Pat. No. 5,972,614; single molecule dilution (SMD) as described in Ruano et al., Proc. Natl. Acad. Sci. 87:6296-300 (1990); and allele specific PCR (Ruaño et al., Nucl. Acids Res. 17:8392 (1989); Ruaño et al., Nucl. Acids Res. 19:6877-82 (1991); Michalatos-Beloin et al., supra).

As will be readily appreciated by those skilled in the art, if the individual is expected to have two alleles for the locus (e.g., the locus is on an autosomal chromosome, or the locus is on the X chromosome and the individual is a female), any individual clone of the locus in that individual will permit directly determining the haplotype for only one of the two alleles; thus, additional clones will need to be examined to directly determine the identity of the haplotype for the other allele. Typically, at least five clones of the genomic locus present in the individual should be examined to have more than a 90% probability of determining both alleles. In some cases, however, once the haplotype for one allele is directly determined, the haplotype for the other allele may be inferred if the individual has a known genotype for the PSs comprising the marker or if the frequency of haplotypes or haplotype pairs for the locus in an appropriate reference population is available.

Direct haplotyping of both alleles may be performed by assaying two hemizygous DNA samples, one for each allele, that are placed in separate containers. Alternatively, the two hemizygous samples may be assayed in the same container if the two samples are labeled with different tags, or if the assay results for each sample are otherwise separately distinguishable or identifiable. For example, if the samples are labeled with first and second fluorescent dyes, and a PS in the locus is assayed using an oligonuclotide probe that is specific for one of the alleles-and labeled with a third fluorescent dye, then detecting a combination of the first and third dyes would identify the nucleotide present at the PS in the first sample while detecting a combination of the second and third dyes would identify the nucleotide present at the PS in the second sample.

Indirect haplotyping methods typically involve preparing a genomic DNA sample isolated from a blood or cheek sample obtained from the individual in a manner that permits accurately determining the individual's genotype for each PS in the locus. The genotype is then used to infer the identity of at least one of the individual's haplotypes for the locus, and preferably used to infer the identity of the individual's haplotype pair for the locus.

In one indirect haplotyping method, the presence of zero, one or two copies of a haplotype of interest can be determined by comparing the individual's genotype for the PS in the marker with a set of reference haplotype pairs for the same set of PS and assigning to the individual a reference haplotype pair that is most likely to exist in the individual. The individual's copy number for the haplotype comprising the marker is how many copies of that haplotype are in the assigned reference haplotype pair.

The reference haplotype pairs are those that are known to exist in the general population or in a reference population or that are theoretically possible based on the alternative alleles possible at each PS. The reference population may be composed of randomly-selected individuals representing the major ethnogeographic groups of the world. A preferred reference population is one having a similar ethnogeographic background as the individual being tested for the presence of the marker. The size of the reference population is chosen based on how rare a haplotype is that one wants to be guaranteed to see. For example, if one wants to have a q % chance of not missing a haplotype that exists in the population at a p % frequency of occurring in the reference population, the number of individuals (n) who must be sampled is given by 2n=log(1−q)/log(1−p) where p and q are expressed as fractions. A particularly preferred reference population includes one or more 3-generation families to serve as a control for checking quality of haplotyping procedures. If the reference population comprises more than one ethnogeographic group, the frequency data for each group is examined to determine whether it is consistent with Hardy-Weinberg equilibrium. Hardy-Weinberg equilibrium (D. L. Hartl et al., Principles of Population Genomics, Sinauer Associates (Sunderland, Mass.), 3d Ed., 1997) postulates that the frequency of finding the haplotype pair H₁/H₂is equal to p_{H-W}(H₁/H₂)=2p(H₁)p(H₂) if H₁≠H₂ and p_{H-W}(H₁/H₂)=p(H₁)p(H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology (U.S. Pat. No. 5,866,404), single molecule dilution, or allele-specific long-range PCR (Michalotos-Beloin et al., Nucleic Acids Res. 24:4841-4843, 1996).

Assignment of the haplotype pair may be performed by choosing a reference haplotype pair that is consistent with the individual's genotype. When the genotype of the individual is consistent with more than one reference haplotype pair, the frequencies of the reference haplotype pairs may be used to determine which of these consistent haplotype pairs is most likely to be present in the individual. If a particular consistent haplotype pair is more frequent in the reference population than other consistent haplotype pairs, then the consistent haplotype pair with the highest frequency is the most likely to be present in the individual. Occasionally, only one haplotype represented in the reference haplotype pairs is consistent with any of the possible haplotype pairs that could explain the individual's genotype, and in such cases the individual is assigned a haplotype pair containing this known haplotype and a new haplotype derived by subtracting the known haplotype from the possible haplotype pair. In rare cases, either no haplotypes in the reference population are consistent with the individual's genotype, or alternatively, multiple reference haplotype pairs are consistent with the genotype. In such cases, the individual is preferably haplotyped using a direct molecular haplotyping method such as, for example, CLASPER System™ technology (U.S. Pat. No. 5,866,404), SMD, or allele-specific long-range PCR (Michalotos-Beloin et al., supra).

Indirect determination of the copy number of haplotypes present in an individual from her genotype is illustrated here for a hypothetical Marker X, which is associated with the adverse hematological response. Marker X consists of one or two copies of Haplotype GA, which contains two polymorphic sites, PSA and PSB, in Gene Y on an autosomal chromosome. The hypothetical below shows the 9 (3ⁿ, where each of n=2 bi-allelic polymorphic sites may have one of 3 different genotypes present) genotypes that may be detected for the set of PSA and PSB, using a genomic DNA sample from an individual. Eight of the nine possible genotypes for the two sites allow unambiguous determination of the number of copies of Haplotype GA present in the individual and therefore would allow unambiguous determination of the presence or absence in the individual of Marker X. However, an individual with the C/G A/C genotype could possess either of the following haplotype pairs: CA/GC or CC/GA, and thus could have either 1 copy of Haplotype GA (CC/GA haplotype pair), which would mean Marker X is present, or 0 copy (CA/GC haplotype pair) of Haplotype GA, which would mean Marker X is absent. For this instance where there is ambiguity in the haplotype pair underlying the determined genotype C/G A/C, frequency information may be used to determine the most probable haplotype pair and therefore the most likely number of copies of the marker haplotype in the individual, as described above. Alternatively, for the ambiguous double heterozygote, genotyping of one or more additional sites in Gene Y or nearby may be performed to resolve this ambiguity. The skilled artisan would recognize that these one or more additional sites would need to have sufficient linkage with the alleles in at least one of the haplotypes in a possible haplotype pair to permit unambiguous assignment of that haplotype pair. Although this illustration has been directed to the particular instance of determining the number of Haplotype AG present in an individual, an analogous process would be used for determining the copy number of any linked or substitute haplotype for Haplotype AG.

### Hypothetical: Possible Copy Numbers of Haplotype (GA)

### Derived from Possible Genotypes at PSA and PSB

| Genotype | | Copy Number of |
|---|---|---|
| PSA | PSB | Haplotype GA |
| G/G | C/C | 0 |
| G/G | A/C | 1 |
| C/G | C/C | 0 |
| C/G | A/C | 1 or 0 |
| G/C | A/A | 1 |
| G/G | A/A | 2 |
| C/C | A/A | 0 |
| C/C | A/C | 0 |
| C/C | C/C | 0 |

Any of all of the steps in the indirect haplotyping method described above may be performed manually, by visual inspection and performing appropriate calculations, but are preferably performed by a computer-implemented algorithm that accesses data on the individual's genotype and reference haplotype pairs stored in computer readable format. Such algorithms are described in WO 01/80156 and PCT/US2004/019023. Alternatively, the haplotype pair in an individual may be predicted from the individual's genotype for that gene with the assistance of other reported haplotyping algorithms (e.g., Clark et al. 1990, Mol Bio Evol 7:111-22; Stephens, M. et al., (2001) Am J Hum Genet 68:978-989; WO 02/064617; Niu T. et al. (2002) Am J. Hum Genet 70:157-169; Zhang et al. (2003) BMC Bioinformatics 4(1):3) or through a commercial haplotyping service such as offered by Genaissance Pharmaceuticals, Inc. (New Haven, Conn.).

All direct and indirect haplotyping methods described herein typically involve determining the identity of at least one of the alleles at a PS in a nucleic acid sample obtained from the individual. To enhance the sensitivity and specificity of that determination, it is frequently desirable to amplify from the nucleic acid sample one or more target regions in the locus. An amplified target region may span the locus of interest, such as an entire gene, or a region thereof containing one or more polymorphic sites. Separate target regions may be amplified for each PS in a marker.

Any amplification technique known to those of skill in the art may be used in practicing the present invention including, but not limited to, polymerase chain reaction (PCR) techniques. PCR may be carried out using materials and methods known to those of skill in the art (See generally PCR Technology: Principals and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Matilla et al., Nucleic Acids Res. 19: 4967 (1991); Eckert et al., PCR Methods and Applications 1: 17 (1991); PCR (eds. McPherson et al., IRL Press, Oxford); and U.S. Pat. No. 4,683,202. Other suitable amplification methods include the ligase chain reaction (LCR) (see Wu and Wallace, Genomics 4: 560 (1989) and Landegren et al., Science 241: 1077 (1988)), transcription amplification (Kwoh et al., Proc. Natl. Acad. Sci. USA 86: 1173 (1989)), self-sustained sequence replication (Guatelli et al., Proc. Nat. Acad. Sci. USA, 87: 1874 (1990)); isothermal methods (Walker et al., Proc. Natl. Acad. Sci. USA 89:392-6 (1992)); and nucleic acid-based sequence amplification (NASBA).

The amplified target region is assayed to determine the identity of at least one of the alleles present at a PS in the region. If both alleles of a locus are represented in the amplified target, it will be readily appreciated by the skilled artisan that only one allele will be detected at a PS in individuals who are homozygous at that PS, while two different alleles will be detected if the individual is heterozygous for that PS. The identity of the allele may be identified directly, known as positive-type identification, or by inference, referred to as negative-type identification. For example, where a SNP is known to be guanine or cytosine in a reference population, a PS may be positively determined to be either guanine or cytosine for an individual homozygous at that site, or both guanine and cytosine, if the individual is heterozygous at that site. Alternatively, the PS may be negatively determined to be not guanine (and thus cytosine/cytosine) or not cytosine (and thus guanine/guanine).

Identifying the allele or pair of alleles at a PS may be accomplished using any technique known to those of skill in the art. Preferred techniques permit rapid, accurate assaying of multiple PS with a minimum of sample handling. Some examples of suitable techniques include, but are not limited to, direct DNA sequencing of the amplified target region, capillary electrophoresis, hybridization of allele-specific probes, single-strand conformation polymorphism analysis, denaturing gradient gel electrophoresis, temperature gradient electrophoresis, mismatch detection; nucleic acid arrays, primer specific extension, protein detection, and other techniques well known in the art. See, for example, Sambrook, et al., Molecular Cloning: A Laboratory Manual (Cold Spring Harbor Laboratory, New York) (2001); Ausubel, et al., Current Protocols in Molecular Biology (John Wiley and Sons, New York) (1997); Orita et al., Proc. Nat. Acad. Sci. 86, 2766-2770 (1989); Humphries et al., in MOLECULAR DIAGNOSIS OF GENETIC DISEASES, Elles, ed., pp. 321-340, 1996; Wartell et al., Nucl. Acids Res. 18:2699-706 (1990); Hsu et al. (1994) Carcinogenesis 15:1657-1662; Sheffield et al., Proc. Natl. Acad. Sci. USA 86:232-6 (1989); Winter et al., Proc. Natl. Acad. Sci. USA 82:7575 (1985); Myers et al. (1985) Nature 313:495; Rosenbaum and Reissner (1987) Biophys Chem 265:12753; Modrich, Ann. Rev. Genet. 25:229-53 (1991); U.S. Pat. No. 6,300,063; U.S. Pat. No. 5,837,832; U.S. Pat. No. 5,459,039; and HuSNP Mapping Assay, reagent kit and user manual, Affymetrix Part No. 90094 (Affymetrix, Santa Clara, Calif.).

In preferred embodiments, the identity of allele(s) at a PS is determined using a polymerase-mediated primer extension method. Several such methods have been described in the patent and scientific literature and include the “Genetic Bit Analysis” method (WO 92/15712) and the ligase/polymerase mediated genetic bit analysis (U.S. Pat. No. 5,679,524. Related methods are disclosed in WO 91/02087, WO 90/09455, WO 95/17676, and U.S. Pat. Nos. 5,302,509 and 5,945,283. Extended primers containing the complement of the polymorphism may be detected by mass spectrometry as described in U.S. Pat. No. 5,605,798. Another primer extension method is allele-specific PCR (Ruaño et al., 1989, supra; Ruaño et al., 1991, supra; WO 93/22456; Turki et al., J. Clin. Invest. 95:1635-41 (1995)). In addition, multiple PSs may be investigated by simultaneously amplifying multiple regions of the nucleic acid using sets of allele-specific primers as described in WO 89/10414.

Another primer extension method for identifying and analyzing polymorphisms employees single-base extension (SBE) of a fluorescently-labeled primer coupled with fluorescence resonance energy transfer (FRET) between the label of the added base and the label of the primer. Typically, the method, such as that described by Chen et al., (Proc. Nat. Acad. Sci. 94:10756-61 (1997)) uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

In all of the above methods, the accuracy and specificity of an assay designed to detect the identity of the allele(s) at any PS is typically validated by performing the assay on DNA samples in which the identity of the allele(s) at that PS is known. Preferably a sample representing each possible allele is included in the validation process. For diploid loci such as those on autosomal and X chromosomes, the validation samples will typically include a sample that is homozygous for the major allele at the PS, a sample that is homozygous for the minor allele at the PS, and a sample that is heterozygous at that PS. These validation samples are typically also included as controls when performing the assay on a test sample (i.e., a sample in which the identity of the allele(s) at the PS is unknown). The specificity of an assay may also be confirmed by comparing the assay result for a test sample with the result obtained for the same sample using a different type of assay, such as by determining the sequence of an amplified target region believed to contain the PS of interest and comparing the determined sequence to a context sequence based on the reference sequence in FIG. 1. The length of the context sequence necessary to establish that the correct genomic position is being assayed will vary based on the uniqueness of the sequence in the target region (for example, there may be one or more highly homologous sequences located in other genomic regions). The skilled artisan can readily determine an appropriate length for a context sequence for any PS using known techniques such as blasting the context sequence against publicly available sequence databases. For amplified target regions, which provide a first level of specificity, examining the context sequence of about 30 to 60 bases on each side of the PS in known samples is typically sufficient to ensure that the assay design is specific for the PS of interest. Occasionally, a validated assay may fail to provide an unambiguous result for a test sample. This is usually the result of the sample having DNA of insufficient purity or quantity, and an unambiguous result is usually obtained by repurifying or reisolating the DNA sample or by assaying the sample using a different type of assay.

Alternatively, the presence or absence of a marker of the invention may be detected by detecting, in a protein sample obtained from the individual, a polypeptide specified by the polymorphism comprising the marker. The polypeptide may be detected using a monoclonal antibody specific for that polypeptide.

Further, in performing any of the methods described herein that require determining the presence or absence of a HLA-C marker of an adverse hematological response to a drug, such determination may be made by consulting a data repository that contains sufficient information on the patient's genetic composition to determine whether the patient has the HLA-C marker. Preferably, the data repository lists what HLA-C marker(s) are present and absent in the individual. The data repository could include the individual's patient records, a medical data card, a file (e.g., a flat ASCII file) accessible by a computer or other electronic or non-electronic media on which appropriate information or genetic data can be stored. As used herein, a medical data card is a portable storage device such as a magnetic data card, a smart card, which has an on-board processing unit and which is sold by vendors such as Siemens of Munich Germany, or a flash-memory card. If the data repository is a file accessible by a computer; such files may be located on various media, including: a server, a client, a hard disk, a CD, a DVD, a personal digital assistant such as a Palm Pilot, a tape, a zip disk, the computer's internal ROM (read-only-memory) or the internet or worldwide web. Other media for the storage of files accessible by a computer will be obvious to one skilled in the art.

IV. Utility of HLA-C Markers of Adverse Drug Response

The phenotypic effect of the HLA-C markers described herein support using these markers in a variety of methods and products, including, but not limited to, diagnostic methods and kits, pharmacogenetic treatment methods, which involve tailoring a patient's drug therapy based on whether the patient has or lacks a genetic marker associated with an adverse hematological response, drug development and marketing, including pre-clinical testing of drugs for their propensity to induce an adverse hematological response, and pharmacogenetic drug products.

The utility of any of the methods or products claimed herein is not dependent on complete correlation between the presence of a genetic marker of the invention and the occurrence of an adverse hematological response, or upon whether a diagnostic method or kit is 100% accurate, or has an specific degree of accuracy, in determining the presence or absence of a genetic marker in every individual, or in predicting for every individual whether the individual is susceptible for an adverse hematological response to a drug. Thus, the inventors herein intend that the terms “determine”, and “determining” and “predicting” should not be interpreted as requiring a definite or certain result; instead these terms should be construed as meaning that a claimed method or kit provides an accurate result for the majority of individuals, or that the result or prediction for any given individual is more likely to be correct than incorrect. Preferably, the accuracy of the result provided by a diagnostic method or kit of the invention is one that a skilled artisan or regulatory authority would consider suitable for the particular application in which the method or kit is used.

Similarly, the utility of the claimed methods of treatment and drug products does not require that they produce the claimed or desired effect in every individual; all that is required is that a clinical practitioner, when applying his or her professional judgment consistent with all applicable norms, decides that the chance of achieving the claimed effect of treating a given individual according to the claimed method or with the claimed drug product is sufficiently high to warrant prescribing the treatment or drug product.

An individual to be tested in, or treated by, any of the methods described herein is a human subject in need of treatment with any of the above described drugs, despite its propensity to induce an adverse hematological response. In some embodiments, the individual has been diagnosed with, or exhibits a symptom of, a disease for which the drug is approved. In other embodiments, the drug is not approved for treating the diagnosed disease or exhibited symptom(s), but the prescribing physician believes the drug may be helpful in treating the individual. In preferred embodiments of the invention, the drug is clozapine and the individual has any psychotic disease or condition for which clozapine has displayed some degree of clinical utility, such as any psychotic disease or psychotic state (including schizophrenia, psychosis secondary to dopaminergic therapy or coexisting psychiatric disorders in Parkinson's disease), affective disorders, personality disorders, dyskinesias and related disorders, dementia, mental retardation or polydipsia/hyponatramia. In more preferred embodiments of the invention, the individual has been diagnosed with schizophrenia, and in particularly preferred embodiments, the individual has been diagnosed with treatment refractory schizophrenia.

A. Diagnostic Methods and Kits

The diagnostic methods and kits of the invention are useful in clinical diagnostic applications as well as in the methods of treatment described below. However, as used herein, the term “diagnostic” is not limited to clinical or medical uses, and that diagnostic methods and kits of the invention claimed herein are also useful in any research application in which it is desirable to test a subject for the presence or absence of any genetic marker described in Section II above. In preferred embodiments, the diagnostic methods and kits of the invention test for, or are designed to test for, respectively, the presence or absence of a set of HLA-C markers, which set may comprise a marker from Table A-1 (Appendix A) for all ethnicities, or a marker from Table A-2 (Appendix A) for Caucasians only, or may comprise all HLA-C markers described herein.

It is contemplated that any or all of the diagnostic methods claimed herein may be performed by a testing laboratory on an individual's biological sample provided directly by the individual or by any third party, such as the individual's physician, a relative of the individual, a person conducting a research study in which the individual is participating and the like. The third party may have a commercial relationship with the testing laboratory, or may be totally independent thereof. Where the results of the diagnostic method is to be used for clinical purposes, the testing laboratory is preferably a clinical laboratory who performs the diagnostic method in compliance with all applicable laws and regulations in the locality where the testing is performed as well as where the individual resides.

In some embodiments, the testing laboratory does not know the identity of the individual whose sample it is testing; i.e., the sample received by the laboratory is anonymized in some manner before being sent to the laboratory. For example, the sample may be merely identified by a number or some other code (a “sample ID”) and the results of the diagnostic method can be reported to the party ordering the test using the sample ID. In preferred embodiments, the link between the identity of an individual and the individual's sample is known only to the individual or to the individual's physician. In other applications, such as research studies, the link may be broken prior to the testing laboratory sending a report of the results; thus, the results cannot be obtained by the individual or the individual's insurance company.

Kits of the invention, which are useful for detecting the presence or absence of a HLA-C marker in an individual, comprise a set of oligonucleotides designed for identifying each of the alleles at each PS in the marker. In preferred embodiments, the set of oligonucleotides is designed to identify the alleles at all polymorphic sites in all of the HLA-C markers described herein. In particularly preferred embodiments, the set of oligonucleotides is designed to identify both alleles at each PS in a set of HLA-C markers, with the marker set comprising a marker from Table A-1 (Appendix A).

In some embodiments, the oligonucleotides in the kit are either allele-specific probes or allele-specific primers. In other embodiments, the kit comprises primer-extension oligonucleotides. In still further embodiments, the set of oligonucleotides is a combination of allele-specific probes, allele-specific primers, or primer-extension oligonucleotides. The kit may comprise oligonucleotides designed for genotyping other PS, which may be in the HLA-C gene or at any other locus of interest in the human genome.

Oligonucleotides in kits of the invention must be capable of specifically hybridizing to a target region of a polynucleotide. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. In some embodiments, the target region contains a PS in a HLA-C marker, while in other embodiments, the target region is located one to 10 nucleotides from the PS.

The composition and length of each oligonucleotide in the kit will depend on the nature of the genomic region containing the PS as well as the type of assay to be performed with the oligonucleotide and is readily determined by the skilled artisan. For example, the polynucleotide to be used in the assay may constitute an amplification product, and thus the required specificity of the oligonucleotide is with respect to hybridization to the target region in the amplification product rather than in genomic DNA isolated from the individual. As another example, if each PS in a HLA-C marker is to be assayed simultaneously, the melting temperatures for the oligonucleotides in the kit will typically be within a narrow range, preferably less than about 5° C. and more preferably less than about 2° C.

In preferred embodiments, each oligonucleotide in the kit is a perfect complement of its target region. An oligonucleotide is said to be a “perfect” or “complete” complement of another nucleic acid molecule if every nucleotide of one of the molecules is complementary to the nucleotide at the corresponding position of the other molecule. While perfectly complementary oligonucleotides are preferred for detecting polymorphisms, departures from complete complementarity are contemplated where such departures do not prevent the molecule from specifically hybridizing to the target region as defined above. For example, an oligonucleotide primer may have a non-complementary fragment at its 5′ end, with the remainder of the primer being completely complementary to the target region. Alternatively, non-complementary nucleotides may be interspersed into the probe or primer as long as the resulting probe or primer is still capable of specifically hybridizing to the target region.

In some preferred embodiments, each oligonucleotide in the kit specifically hybridizes to its target region under stringent hybridization conditions. Stringent hybridization conditions are sequence-dependent and vary depending on the circumstances. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. As the target sequences are generally present in excess, at Tm, 50% of the probes are occupied at equilibrium. Typically, stringent conditions include a salt concentration of at least about 0.01 to 1.0 M Na ion concentration (or other salts) at pH 7.0 to 8.3 and the temperature is at least about 25° C. for short oligonucleotide probes (e.g., 10 to 50 nucleotides). Stringent conditions can also be achieved with the addition of destabilizing agents such as formamide. For example, conditions of 5× SSPE (750 mM NaCl, 50 mM NaPhosphate, 5 mM EDTA, pH 7.4) and a temperature of 25-30° C. are suitable for allele-specific probe hybridizations. Additional stringent conditions can be found in Molecular Cloning: A Laboratory Manual, Sambrook et al., Cold Spring Harbor Press, Cold Spring Harbor, N.Y. (1989), chapters 7, 9, and 11, and in NUCLEIC ACID HYBRIDIZATION, A PRACTICAL APPROACH, Haymes et al., IRL Press, Washington, D.C., 1985.

A preferred, non-limiting example of stringent hybridization conditions includes hybridization in 4X sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1× SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41 (% G+C)−(600/N), where N is the number of bases in the hybrid, and [Na⁺] is the concentration of sodium ions in the hybridization buffer ([Na⁺] for 1×SSC=0.165 M).

The oligonucleotides in kits of the invention may be comprised of any phosphorylation state of ribonucleotides, deoxyribonucleotides, and acyclic nucleotide derivatives, and other functionally equivalent derivatives. Alternatively, the oligonucleotides may have a phosphate-free backbone, which may be comprised of linkages such as carboxymethyl, acetamidate, carbamate, polyamide (peptide nucleic acid (PNA)) and the like (Varma, in MOLECULAR BIOLOGY AND BIOTECHNOLOGY, A COMPREHENSIVE DESK REFERENCE, Meyers, ed., pp. 617-20, VCH Publishers, Inc., 1995). The oligonucleotides may be prepared by chemical synthesis using any suitable methodology known in the art, or may be derived from a biological sample, for example, by restriction digestion. The oligonucleotides may contain a detectable label, according to any technique known in the art, including use of radiolabels, fluorescent labels, enzymatic labels, proteins, haptens, antibodies, sequence tags and the like. The oligonucleotides in the kit may be manufactured and marketed as analyte specific reagents (ASRs) or may be constitute components of an approved diagnostic device.

In some embodiments, the set of oligonucleotides in the kit have different labels to allow determining the identity of the alleles at two or more PSs simultaneously. The oligonucleotides may also comprise an ordered array that is immobilized on a solid surface such as a microchip, bead, or glass slide (see, e.g., WO 98/20020 and WO 98/20019). Kits comprising such immobilized oligonucleotides may be designed to perform a variety of polymorphism detection assays, including but not limited to probe hybridization and polymerase extension assays.

Kits of the invention may also contain other reagents such as hybridization buffer (e.g., where the oligonucleotides are to be used as allele-specific probes) or dideoxynucleotide triphosphates (ddNTPs; e.g., where the alleles at the polymorphic sites are to be detected by primer extension). Kits designed for use in polymerase-mediated genotyping assays, may also contain a polymerase and a reaction buffer optimized for the polymerase-mediated assay to be performed. Kits of the invention may also include reagents to detect when a specific hybridization has occurred or a specific polymerase-mediated extension has occurred. Such detection reagents may include biotin- or fluorescent-tagged oligonucleotides or ddNTPs and/or an enzyme-labeled antibody and one or more substrates that generate a detectable signal when acted on by the enzyme. It will be understood by the skilled artisan that the set of oligonucleotides and reagents for performing the assay will be provided in separate receptacles placed in the kit container if appropriate to preserve biological or chemical activity and enable proper use in the assay.

In other preferred embodiments, each of the oligonucleotides and all other reagents in the kit have been quality tested for optimal performance in an assay designed to determine each of the alleles at the set of PSs comprising a marker a HLA-C marker. In more preferred embodiments, the kit includes an instruction manual that describes the various ways the kit may be used to detect the presence or absence of a HLA-C marker.

In some preferred embodiments, the set of oligonucleotides in the kit are allele-specific oligonucleotides. As used herein, the term allele-specific oligonucleotide (ASO) means an oligonucleotide that is able, under sufficiently stringent conditions, to hybridize specifically to one allele of a PS, at a target region containing the PS while not hybridizing to the same region containing a different allele. As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes and primers are found in Kogan et al., “Genetic Prediction of Hemophilia A” in PCR PROTOCOLS, A GUIDE TO METHODS AND APPLICATIONS, Academic Press, 1990, and Ruaño et al., Proc. Natl. Acad. Sci. USA 87:6296-300 (1990).

Typically, an ASO will be perfectly complementary to one allele while containing a single mismatch for another allele. In ASO probes, the single mismatch is preferably within a central position of the oligonucleotide probe as it aligns with the polymorphic site in the target region (e.g., approximately the 7^{th} or 8^{th} position in a 15mer, the 8^{th} or 9^{th} position in a 16mer, and the 10^{th} or 11^{th} position in a 20mer). The single mismatch in ASO primers is located at the 3′ terminal nucleotide, or preferably at the 3′ penultimate nucleotide. ASO probes and primers hybridizing to either the coding or noncoding strand are contemplated by the invention.

In other preferred embodiments, the kit comprises a pair of allele-specific oligonucleotides for each PS to be assayed, with one member of the pair being specific for one allele and the other member member being specific for the other allele. In such embodiments, the oligonucleotides in the pair may have different lengths or have different detectable labels to allow the user of the kit to determine which allele-specific oligonucleotide has specifically hybridized to the target region, and thus determine which allele is present in the individual at the assayed PS.

In still other preferred embodiments, the oligonucleotides in the kit are primer-extension oligonucleotides. Termination mixes for polymerase-mediated extension from any of these oligonucleotides are chosen to terminate extension of the oligonucleotide at the PS of interest, or one base thereafter, depending on the alternative nucleotides present at the PS.

B. Pharmacogenetic Treatment Methods

The HLA-C markers of the invention are useful for helping physicians make decisions about how to treat an individual who is a candidate for treatment with a drug that has a propensity for inducing an adverse hematological response.

For example, if the patient has a HLA-C marker, the physician may decide to not treat the individual with the drug or alternatively, decide to treat the individual with the drug but in conjunction with monitoring the individual's neutrophil count for onset of the adverse hematological response. This monitoring process would typically include determining the individual's baseline neutrohpil count prior to administering the drug and then determining the individual's neutrophil count at frequent intervals during treatment with the drug. The frequency of these determinations would be as often as the physician believes is prudent, and could for example, include a consideration of what type of precaution or warning is present on the label for the drug with respect to the adverse hematological response. For example, the individual's neutrophil count could be determined as infrequently as once a month, or at more frequent intervals such as every two weeks or every week. If the patient lacked any HLA-C marker, then the physician may decide to treat the individual with the drug with no monitoring or check the individual's neutrophil count on a less frequent basis and or for a shorter time period than typically recommended for that drug.

In some embodiments, if a HLA-C marker is present, then the physician may decide to treat the patient with the drug in combination with an agent capable of stimulating the production of neutrophils. For example, cases of drug-induced neutropenia and other types of acquired neutropenia have been effectively treated with granulocyte colony stimulating factor (G-CSF), a cytokine which plays an essential role in neutrophil hematopoiesis (Berliner et al., supra; Barreda et al., supra). Another cytokine, granulocyte-macrophage colony-stimulating factor (GM-CSF), which is involved in the upregulation of hematopoietic development, has been used clinically in treating chemotherapy-induced neutropenia and in stimulating hematopoiesis following bone marrow transplantation. (See, e.g., Barreda et al., supra and references cited therein.) G-CSF and GM-CSF are members of a select group of cytokines that promote the development of early hematopoietic cells into cells of the myeloid, lymphoid and erythroid lineages. Other members of this group include interleukin-3 (IL-3, also known as multi-colony stimulating factor), stem cell factor (SCF or c-kit ligand), erythropoietin (EPO) and others. IL-3 acts on earlier progenitors than GM-CSF, and supports colony formation by multi-lineage, granulocyte-macrophage, and granulocyte cells. Thus, a physician might choose to co-administer with the drug one or more cytokines that support the growth and maturation of neutrophil progenitors, such as G-CSF, GM-CSF and IL-3.

In other embodiments, the physician may decide to treat a patient who has a HLA-C marker by coadministering the drug with an agent capable of inhibiting the induction of the adverse hematological response. This agent could function by blocking a mechanism involved in the etiology of the adverse hematological response.

For example, U.S. Pat. No. 5,312,819 suggests that clozapine-induced agranulocytosis is caused by reactive free radicals of clozapine or a metabolite thereof and teaches that the radical scavenger L-ascorbic acid reduces the formation of free radicals of clozapine. WO 93/08801 extends this free radical theory to explain granulocytopenia and agranulocytosis induced by other drugs, and showed that L-ascorbic acid reduces the formation of free radicals of a number of drugs known or suspected to cause such hematological toxicity. WO 93/08801 proposes that such hematological toxicity is caused by the oxidation of an active drug or a metabolite by myeloperoxidase released by activated neutrophils. U.S. Pat. No. 5,312,819 and WO 93/08801 conclude that co-administration of a radical scavenger with clozapine, or another drug that can induce agranulocytosis, respectively, would inhibit the induction of granulocytopenia and agranulocytosis. Particular radical scavengers claimed by U.S. Pat. No. 5,312,819 as being effective for this purpose are L-ascorbic acid, L-ascorbic acid 6-palmitate, ubiquinol-10 and α-tocopherol, with recommended dosages of L-ascorbic acid ranging between 0.5 and 20 g or based on administering a weight ratio of clozapine to L-ascorbic acid of about 1:3 to 1:40.

In preferred embodiments, if the individual is a candidate for clozapine treatment, and tests negative for a HLA-C marker, then the physician may be more likely to decide to prescribe clozapine than if the physician had no information on whether the marker was present or absent. However, under the current distribution system for clozapine drug products that exists in the United States, the physician would still be required to conduct the WBC monitoring process set forth in the labels of these drug products, as exemplified by the currently approved lable for clozapine drug products. However, if a clozapine drug product is approved for a pharmacogenetic indication as described in Section IV-C or Section IV-D, then it is contemplated that a physician would choose that clozapine drug product to treat individuals who are within the approved pharmacogenetic indication.

In preferred embodiments, each of the above pharmacogenetic treatment methods of the invention involve determining the presence or absence in an individual of a set of HLA-C markers, which set may comprise a marker from Table A-1 (Appendix A), or may comprise all HLA-C markers described herein.

C. Pharmacogenetic Drug Development and Marketing

The inventors herein contemplate that the HLA-C markers described herein could be used to seek regulatory approval to marker a new clozapine drug product that is indicated for patients lacking any HLA-C marker and who are able to comply with a WBC monitoring process that is less rigorous than the process described in the currently approved label for clozapine drug products. Indeed, since the current distribution system for clozapine drug products is unique among drugs that have warnings of a risk for an adverse hematological response, it is contemplated that WBC monitoring could be eliminated entirely for a clozapine drug product indicated only for patients who test negative for a combination of genetic markers associated with the adverse hematological response (e.g., a “pharmacogenetic indication”), if such combination identifies a sufficient percentage of those individuals who are genetically predisposed for this side effect.

Similarly, the inventors herein contemplate that HLA-C markers described herein are useful for seeking approval of pharmacogenetic indications for currently approved drugs that physicians are reluctant to prescribe because of their propensity to induce an adverse hematological response. More broadly, the HLA-C markers described herein could be used in seeking approval to market drugs in development which appear to have an unacceptable risk of an adverse hematological response in the general population. These pharmacogenetic drug development methods could increase the use of highly effective drugs that are currently underutilized due to this safety concern, or make available for a certain population drugs for which additional therapies are needed, but that might otherwise not be approved.

Seeking approval for a pharmacogenetic indication of a drug with a known propensity to induce an adverse hematological response typically involves measuring the incidence of the adverse hematological response in two separate groups of patients treated with the drug. Preferably, all individuals in both groups have been diagnosed with a disease for which the drug has demonstrated efficacy. Each individual within one of the groups has a genetic profile that places the individual within the proposed pharmacogenetic indication. The individuals in the other group may be randomly selected without regard to whether they meet the proposed pharmacogenetic indication. Alternately, the individuals are assigned to the other group in a manner that results in a “control” group in which the percentage of individuals who meet and do not meet the pharmacogenetic indication is similar to what is observed in the general population, or in a population of patients with the disease that the drug has efficacy. The drug product for which approval is sought could be administered to the two groups in a prospective trial. Alternatively, a retrospective pharmacogenetic analysis of patients previously treated with the drug could be performed, a route which may be necessary if the mortality risk of the adverse hematological response is sufficiently high to make a prospective trial unethical.

The drug product tested in a prospective or retrospective pharmacogenetic trial may contain other active ingredients, for example another drug with efficacy for treating the disease or condition in the proposed pharmacogenetic indication or an agent that is intended to reduce the incidence of a different side effect caused by the drug. In some embodiments, the pharmacogentic indication for which regulatory approval is sought comprises the absence of all HLA-C markers described herein. In preferred embodiments, the pharmacogentic indication being sought further comprises the absence of all other known genetic markers associated with the adverse hematological response.

The pharmacogenetic study could be designed in consultation with representatives of the regulatory agency or government entity from whom approval is required before marketing the pharmacogenetic drug product in a particular country. Preferably, the regulatory agency is authorized by the government of a major industrialized country, such as Australia, Canada, China, a member of the European Union, Japan, and the like. Most preferably the regulatory agency is authorized by the government of the United States and the type of application for approval that is filed will depend on the legal requirements set forth in the last enacted version of the Food, Drug and Cosmetic Act that are applicable for the drug product and may also include other considerations such as the cost of making the regulatory filing and the marketing strategy for the drug product. For example, if the pharmaceutical formulation in the drug product has previously been approved for the same disease indication, then the application might be a paper NDA, a supplemental NDA or an abbreviated NDA, but the application would be a full NDA if the pharmaceutical formulation has never been approved before; with these terms having the meanings applied to them by those skilled in the pharmaceutical arts or as defined in the Drug Price Competition and Patent Term Restoration Act of 1984.

In other embodiments, drugs in the pre-clinical phase of development may be tested for their propensity to induce an adverse hematological response using in vitro assays that assess the effect of the drug of interest on cells that express a polypeptide encoded by an allele of the HLA-C gene that is specified by the polymorphism in a HLA-C marker.

D. Pharmacogenetic Drug Products

One desired outcome of a pharmacogenetic clinical trial using the HLA-C markers such as described above is approval to market a drug product which comprises a drug associated with an adverse hematological response in patients who have a HLA-C marker and prescribing information which includes an approved indication for the drug product. The approved indication has two parts: a disease indication and a pharmacogenetic indication. The disease indication is description of the disease or condition for which the drug has demonstrated efficacy and the pharmacogentic indication is the absence of a HLA-C marker. In preferred embodiments, the pharmacogenetic indication is the absence of all HLA-C markers described herein and more preferably the absence of all known genetic markers associated with the adverse hematological response. It is intended that the pharmacogenetic indication may be equivalently provided by the prescribing information stating that the drug product is contraindicated in patients having a HLA-C marker. As described above, pharmacogenetic drug products of the invention may include an additional active ingredient with demonstrated efficacy for the disease indication or that is capable of inhibiting the adverse hematological response. The drug product may also contain additional approved indications that include a different disease indication and the same or a different pharmacogenetic indication.

The drug may be formulated in any way known in the art, for any mode of delivery (e.g., oral, transdermal) and any mode of release (e.g., sustained release). The formulation selected will depend on the characteristics of the drug and the intended disease indication(s), and my be readily determined by those skilled in the art. In some embodiments, the formulation has a distinctive appearance that the manufacturer has adopted to identify the drug product as a pharmacogenetic product to aid pharmacists and physicians in distinguishing this product from other marketed products comprising the drug, but which do not have a pharmacogenetic indication. Using the appearance of pharmaceutical formulations as part of creating a distinctive brand for drug products is well known in the art, and includes the shape and color of tablets or capsules, as well as symbols or logos stamped thereon, or on the packaging material in which the formulation is distributed or sold.

Preferred pharmacogenetic drug products of the invention comprise clozapine and prescribing information which provides instructions for performing a prescribing process to determine whether a patient may be initially prescribed the clozapine drug product. The prescribing process comprises determining whether the patient has a genetic profile within the pharmacogenetic indication, obtaining a baseline white blood cell count (WBC) and using the results of the first two steps to make a prescribing decision. The drug is prescribed only if the patient's genetic profile is within the pharmacogenetic indication and has a baseline WBC of at least 3500/mm³. In preferred embodiments, if the clozapine drug product is prescribed, the prescribing process further comprises monitoring the white blood cell counts according to a schedule to determine whether treatment with the drug product should be interrupted or discontinued. In more preferred embodiments, this prescribed monitoring schedule is less rigorous than the monitoring schedule set forth in the currently approved label for clozapine drug products. Preferably, a less rigorous monitoring schedule requires a fewer total number of WBC counts during clozapine therapy. For example, the prescribed monitoring schedule in the currently approved label for clozapine drug products requires weekly WBC counts during the first 6 months of continuous treatment (the “first treatment period”), and every other week thereafter (the “second treatment period”). The prescribed monitoring schedule for a preferred clozapine drug product of the invention may require white blood cell counts at the same interval as in the first or second treatment period, but for a shorter time, or may require WBC counts at less frequent intervals in the first or second treatment periods, but for the same length of time. In some preferred embodiments, the prescribed monitoring schedule includes interrupting or discontinuing treatment with the clozapine drug product if the patient's WBC count falls below one or more of the WBC thresholds set forth in the currently approved label for clozapine drug products.

Any or all analytical and mathematical operations involved in performing the methods described herein or in using the kits and products described herein may be implemented by a computer. For example, the computer may execute a program that assigns the presence or absence of a HLA-C marker to an individual based on genotype data inputted by an employee of a testing laboratory or by the treating physician. In addition, the computer may output the predicted hematological response to a drug using the individual's genotype data for the polymorphic sites in a HLA-C marker, which may have been determined by the same or different computer program or input by the testing laboratory or the treating physician. Data relating to the presence or absence of HLA-C markers in an individual may be stored as part of a relational database (e.g., an instance of an Oracle database or a set of ASCII flat files) containing other clinical and/or genetic data for the individual. These data may be stored on the computer's hard drive or may, for example, be stored on a CD ROM or on one or more other storage devices accessible by the computer. For example, the data may be stored on one or more databases in communication with the computer via a network.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims that follow the examples.

### EXAMPLES

The Examples herein are meant to exemplify the various aspects of carrying out the invention and are not intended to limit the scope of the invention in any way. The Examples do not include detailed descriptions for conventional methods employed, such as the design of PCR primers, performing PCR, and haplotyping. Such methods are well known to those skilled in the art and are described herein or in numerous publications, for example, MOLECULAR CLONING: A LABORATORY MANUAL, 2^{nd} ed., supra.

### Example 1

This example illustrates the inclusion and exclusion criteria in a case-control study to detect genetic markers associated with clozapine-induced agranulocytosis. The inclusion criteria for the cases were (1) an age of 18-75, (2) a diagnosis of agranulocytosis (absolute neutrophil count of less than 500/mm³) during treatment with clozapine, and (3) a discontinuance of treatment with clozapine at the time of the diagnosis. The inclusion criteria for the controls were (1) an age of 18-75 and (2) treatment with at least 250 mg of clozapine for at least twelve months without a reduction in white blood cell count to less than 3000/mm³ or a reduction in absolute neutrophil count to less than 1500/mm³. The exclusion criteria for both cases and controls were (1) current enrollment in an investigational drug study, (2) compromised or suppressed immunity, and (3) known bone marrow disease. The covariates were age, gender, and ethnicity. The total numbers for the study were 33 cases (28 Caucasian) and 54 controls (48 Caucasian).

### Example 2

This example illustrates genotyping of the study group for the ten HLA-C polymorphic sites selected by the inventors herein for analysis. Genomic DNA samples were isolated from blood samples obtained from each individual and amplified target regions containing the polymorphic sites in Table A-2 (Appendix A) were sequenced to determine the study subjects' genotypes at these polymorphic sites. Tailed (Universal M13 Forward and Reverse) PCR primers were designed using the sequence of SEQ ID NO:1. Amplified PCR products were sequenced using Applied Biosystems' Big Dye® Terminator v 3.1 cycle sequencing kit according to manufacturer's instructions. The reaction products were then electrophoresed using an Applied Biosystems 3700 or 3730×1 DNA analyzer. Polymorphisms were identified using the Polyphred program, and confirmed by visual inspection.

### Example 3

This example illustrates the deduction of markers from the HLA-C genotyping data generated in Example 2.

Haplotypes were estimated from the unphased genotypes using a computer-implemented algorithm for assigning haplotypes to unrelated individuals in a population sample, essentially as described in WO 01/80156 (Genaissance Pharmaceuticals, Inc., New Haven, Conn.). In this method, haplotypes are assigned directly from individuals who are homozygous at all sites or heterozygous at no more than one of the variable sites. This list of haplotypes is then used to deconvolute the unphased genotypes in the remaining (multiply heterozygous) individuals.

A quality control analysis was performed on the deduced haplotypes, which included analysis of the frequencies of the haplotypes and individual SNPs therein for compliance with principles of Hardy-Weinberg equilibrium.

### Example 4

This example illustrates analysis of the HLA-C markers in Table A-1 (Appendix A) for association with hematological response to clozapine. A proprietary algorithm was used as a tool for finding associations between markers and outcomes. The clinical outcome was agranulocytosis case status. A linear model was fitted on the covariates. The resulting residuals were used as the outcome in a t-test in which, for each haplotype being considered, the dominant or recessive mode divided the sample into two groups.

For the results obtained on the analyses, adjustments were made for multiple comparisons, using a permutation test (PERMUTATION TESTS: A PRACTICAL GUIDE TO RESAMPLING METHODS FOR TESTING HYPOTHESES, 2^{nd} ed., Good, Springer Series in Statistics, New York, 2000). In this test, the marker data for each observation were kept constant, while all the remaining variables (outcome and covariates) were randomly permuted so that covariates always stayed with the same outcome. The permutation model was fitted for each of the several haplotypes, and the lowest p-value was kept. In total, up to 6250 permutations were done, depending on the level of significance. Fourteen markers were identified that show a correlation with clozapine-induced agranulocytosis. The unadjusted (“raw”) and adjusted p-values for these markers are shown in Table 1.

TABLE 1

| | | Adjusted | Crude | Marker | | No Marker | |
|---|---|---|---|---|---|---|---|
| Marker | Raw p-value | p-value | Odds Ratio | Cases | Controls | Cases | Controls |
| 1 | 0.000016 | 0.063096 | 7.07 | 27 | 21 | 6 | 33 |
| 2 | 0.000016 | 0.079433 | 7.07 | 27 | 21 | 6 | 33 |
| 3 | 0.000032 | 0.1 | 5.98 | 23 | 15 | 10 | 39 |
| 4 | 5.8E−06 | 4.8E−02 | 7.65 | 27 | 20 | 6 | 34 |
| 5 | 7.5E−06 | 5.6E−02 | 7.65 | 27 | 20 | 6 | 34 |
| 6 | 7.6E−06 | 5.6E−02 | 8.14 | 28 | 22 | 5 | 32 |
| 7 | 8.2E−06 | 5.6E−02 | 7.65 | 27 | 20 | 6 | 34 |
| 8 | 9.8E−06 | 7.2E−02 | 8.15 | 28 | 22 | 5 | 32 |
| 9 | 1.1E−05 | 7.2E−02 | 8.15 | 28 | 22 | 5 | 32 |
| 10 | 2.0E−05 | 1.0E−01 | 7.55 | 28 | 23 | 5 | 31 |
| 11 | 2.1E−05 | 1.0E−01 | 7.55 | 28 | 23 | 5 | 31 |
| 12 | 2.2E−05 | 1.1E−01 | 7.07 | 27 | 21 | 6 | 33 |
| 13 | 2.7E−05 | 1.1E−01 | 6.13 | 21 | 12 | 12 | 42 |
| 14 | 2.7E−05 | 1.1E−01 | 7.55 | 28 | 23 | 5 | 31 |

In view of the above, it will be seen that the several advantages of the invention are achieved and other advantageous results attained. As various changes could be made in the above methods and compositions without departing from the scope of the invention, it is intended that all matter contained in the above description and shown in the accompanying drawings shall be interpreted as illustrative and not in a limiting sense.

All references cited in this specification, including patents and patent applications, are hereby incorporated in their entirety by reference. The discussion of references herein is intended merely to summarize the assertions made by their authors and no admission is made that any reference constitutes prior art. Applicants reserve the right to challenge the accuracy and pertinence of the cited references.

In view of the above, it will be seen that the several advantages of the invention are achieved and other advantageous results attained. As various changes could be made in the above methods and compositions without departing from the scope of the invention, it is intended that all matter contained in the above description and shown in the accompanying drawings shall be interpreted as illustrative and not in a limiting sense.

All references cited in this specification, including patents and patent applications, are hereby incorporated in their entirety by reference. The discussion of references herein is intended merely to summarize the assertions made by their authors and no admission is made that any reference constitutes prior art. Applicants reserve the right to challenge the accuracy and pertinence of the cited references.

TABLE A-1
HLA-C Markers Associated with Adverse Hematological Response to
Clozapine

| Marker | Polymorphism | Copy No. of Polymorphism |
|---|---|---|
| 1 | PS3-G, PS41-G, PS54-G | 1 or 2 |
| 2 | PS3-G, PS12-C, PS87-A | 1 or 2 |
| 3 | PS10-C, PS41-G, PS54-G | 1 or 2 |
| 4 | PS3-G, PS12-C, PS41-G, PS87-A | 1 or 2 |
| 5 | PS3-G, PS41-G, PS54-G, PS70-A | 1 or 2 |
| 6 | PS12-C, PS41-G, PS69-G, PS87-A | 1 or 2 |
| 7 | PS3-G, PS12-C, PS70-A, PS87-A | 1 or 2 |
| 8 | PS41-G, PS54-G, PS69-G, PS70-A | 1 or 2 |
| 9 | PS12-C, PS69-G, PS70-A, PS87-A | 1 or 2 |
| 10 | PS41-G, PS54-G, PS69-G | 1 or 2 |
| 11 | PS12-C, PS69-G, PS87-A | 1 or 2 |
| 12 | PS3-G, PS6-G, PS54-G, PS70-A | 1 or 2 |
| 13 | PS3-G, PS54-G, PS55-G, PS70-A | 1 or 2 |
| 14 | PS6-G, PS54-G, PS69-G, PS70-A | 1 or 2 |

TABLE A-2
Polymorphic Sites in the HLA-C Gene

| | | Position | | |
|---|---|---|---|---|
| | | in FIG. | | |
| PS | | 1/SEQ ID | Reference | Variant |
| Number | Poly ID¹ | NO: 1 | Allele | Allele |
| 3 | 277543433 | 1079 | G | T |
| 6 | 196640272 | 1304 | G | A |
| 10 | 196640229 | 1372 | T | C |
| 12 | 196640189 | 1422 | C | T |
| 41 | 196639975 | 1816 | G | T |
| 54 | 191281890 | 2002 | G | A |
| 55 | 191281899 | 2015 | G | A |
| 69 | 54098889 | 3055 | G | C |
| 70 | 54098885 | 3087 | A | G |
| 87 | 277550795 | 3423 | A | G |

¹The Poly ID is a unique identifier assigned to the indicated PS by Genaissance Pharmaceuticals, Inc., New Haven, CT.

What is claimed is:

1. A method of testing an individual for susceptibility for an adverse hematological response to treatment with a drug, the method comprising:
(a) detecting, in a biological sample obtained from the individual, the presence or absence in the individual of a genetic marker in the HLA-C gene that is associated with the hematological adverse response; and
(b) generating a test report for the individual, wherein if the genetic marker is present in the individual, then the test report indicates that the individual is susceptible for the adverse hematological response, and if the genetic marker is not present in the individual, then the test report indicates that the individual is not susceptible for the hematological adverse response.

2. A method of testing an individual for the presence or absence of a genetic marker that is associated with an adverse hematological response to treatment with a drug, the method comprising:
(a) determining, for a biological sample obtained from the individual, the copy number of a polymorphism in the HLA-C gene that is associated with the adverse hematological adverse response;
(b) using the determined copy number to assign to the individual the presence or absence of the genetic marker; and
(c) generating a test report which indicates whether the genetic marker is present or absent in the individual.

3. A method of predicting whether an individual is susceptible for a hematological adverse response to treatment with a drug, the method comprising:
(a) determining the presence or absence in the individual of a genetic marker in the HLA-C gene that is associated with the hematological adverse response; and
(b) making a prediction based on the results of the determining step, wherein if the HLA-C marker is present, then the prediction is that the individual is likely to exhibit the hematological adverse response if treated with the drug and if the HLA-C marker is absent, the prediction is that the individual is not likely to exhibit the hematological adverse response.

4. A kit for detecting a genetic marker in the HLA-C gene that is associated with an adverse hematological response to treatment with a drug, the kit comprising a set of oligonucleotides designed for identifying each of the alleles at each polymorphic site (PS) in the HLA-C marker.

5. The kit of claim 4, wherein the set of oligonucleotides comprises an allele-specific oligonucleotide (ASO) probe for each allele at each PS.

6. The kit of claim 4, wherein the set of oligonucleotides comprises a primer-extension oligonucleotide for each PS.

7. The method of claim 1, wherein the drug is an antithyroid medication.

8. The method of claim 2, wherein the drug is an antithyroid medication.

9. The method of claim 3, wherein the drug is an antithyroid medication.

10. The kit of claim 4, wherein the drug is an antithyroid medication.

11. The method of claim 1, wherein the drug is a sulfonamide.

12. The method of claim 2, wherein the drug is a sulfonamide.

13. The method of claim 3, wherein the drug is a sulfonamide.

14. The kit of claim 4, wherein the drug is a sulfonamide.

15. The method of claim 1, wherein the label of the drug comprises a warning that the drug is associated with a risk for neutropenia or agranulocytosis.

16. The method of claim 2, wherein the label of the drug comprises a warning that the drug is associated with a risk for neutropenia or agranulocytosis.

17. The method of claim 3, wherein the label of the drug comprises a warning that the drug is associated with a risk for neutropenia or agranulocytosis.

18. The kit of claim 4, wherein the label of the drug comprises a warning that the drug is associated with a risk for neutropenia or agranulocytosis.

19. The method of claim 1, wherein the drug is any of the following compounds or a pharmaceutically acceptable salt thereof: (1) clozapine; (2) quinapril; (3) moexipril; (4) benazepril; (5) enalapril; (6) perindopril erbumine; (7) carbamazepine; (9) lisinopril; (10) trandolapril; (11) ticlopidine; (12) captotril; (13) benazepril; (14) ramipril; (15) penicillamnine; (16) propafenone; (17) sulfamethoxazole; (18) zonisamide; (19) leflunomide; (20) sulfacetamide; (21) prednisolone; (22) timolol; (23) dapsone; (24) ofloxacin; (25) levofloxacin; (26) sulfisoxazole; (27) promethazine; (28) amoxicillin; (29) mebendazole; (30) brinzolamide; (31) procainamide and (32) tocainide.

20. The method claim 2, wherein the drug is any of the following compounds or a pharmaceutically acceptable salt thereof: (1) clozapine; (2) quinapril; (3) moexipril; (4) benazepril; (5) enalapril; (6) perindopril erbumine; (7) carbamazepine; (9) lisinopril; (10) trandolapril; (11) ticlopidine; (12) captotril; (13) benazepril; (14) ramipril; (15) penicillamine; (16) propafenone; (17) sulfamethoxazole; (18) zonisamide; (19) leflunomide; (20) sulfacetamide; (21) prednisolone; (22) timolol; (23) dapsone; (24) ofloxacin; (25) levofloxacin; (26) sulfisoxazole; (27) promethazine; (28) amoxicillin; (29) mebendazole; (30) brinzolamide; (31) procainamide and (32) tocainide.

21. The method of claim 3, wherein the drug is any of the following compounds or a pharmaceutically acceptable salt thereof: (1) clozapine; (2) quinapril; (3) moexipril; (4) benazepril; (5) enalapril; (6) perindopril erbumine; (7) carbamazepine; (9) lisinopril; (10) trandolapril; (11) ticlopidine; (12) captotril; (13) benazepril; (14) ramipril; (15) penicillamine; (16) propafenone; (17) sulfamethoxazole; (18) zonisamide; (19) leflunomide; (20) sulfacetamide; (21) prednisolone; (22) timolol; (23) dapsone; (24) ofloxacin; (25) levofloxacin; (26) sulfisoxazole; (27) promethazine; (28) amoxicillin; (29) mebendazole; (30) brinzolamide; (31) procainamide and (32) tocainide.

22. The kit of claim 4, wherein the drug is any of the following compounds or a pharmaceutically acceptable salt thereof: (1) clozapine; (2) quinapril; (3) moexipril; (4) benazepril; (5) enalapril; (6) perindopril erbumine, (7) carbamazepine; (9) lisinopril; (10) trandolapril; (11) ticlopidine; (12) captotril; (13) benazepril; (14) ramipril; (15) penicillamine; (16) propafenone; (17) sulfamethoxazole; (18) zonisamide; (19) leflunomide; (20) sulfacetamide; (21) prednisolone; (22) timolol; (23) dapsone; (24) ofloxacin; (25) levofloxacin; (26) sulfisoxazole; (27) promethazine; (28) amoxicillin; (29) mebendazole; (30) brinzolamide; (31) procainamide and (32) tocainide.

23. The method of claim 1, wherein the drug is any of the following compounds or a pharmaceutically acceptable salt thereof: clozapine, carbamazepine, ticlopidine, procainamide or tocainide.

24. The method of claim 2, wherein the drug is any of the following compounds or a pharmaceutically acceptable salt thereof: clozapine, carbamazepine, ticlopidine, procainamide or tocainide.

25. The method of claim 3, wherein the drug is any of the following compounds or a pharmaceutically acceptable salt thereof: clozapine, carbamazepine, ticlopidine, procainamide or tocainide.

26. The kit of claim 4, wherein the drug is any of the following compounds or a pharmaceutically acceptable salt thereof: clozapine, carbamazepine, ticlopidine, procainamide or tocainide.

27. The method of claim 1, wherein the drug is clozapine.

28. The method of claim 2, wherein the drug is clozapine.

29. The method of claim 3, wherein the drug is clozapine.

30. The kit of claim 4, wherein the drug is clozapine.

31. A method of selecting a suitable therapy for an individual who is a candidate for treatment with a drug that has a propensity for inducing an adverse hematological response, the method comprising:
(a) determining the presence or absence in the individual of a genetic marker in the HLA-C gene that is associated with the adverse hematological response, and
(b) selecting the therapy based on the results of the determining step, wherein if the HLA-C marker is determined to be absent in the individual, the selected therapy comprises treating the individual with the drug.

32. The method of claim 31, wherein if the HLA-C marker is determined to be present in the individual, the selected therapy comprises treating the individual with a drug that is not known to induce an adverse hematological response.

33. The method of claim 31, wherein if the HLA-C marker is determined to be present in the individual, the selected therapy comprises treating the individual with the drug and monitoring the individual's neutrophil count for onset of the adverse hematological response.

34. The method of claim 31, wherein the selected therapy comprises co-administering to the individual the drug and a cytokine composition in an amount effective to stimulate the production of neutrophils, wherein the cytokine composition comprises one or more of G-CSF, GM-CSF, and IL-3.

35. The method of claim 31, wherein the selected therapy comprises co-administering to the individual the drug and a radical scavenger in an amount effective to inhibit the adverse hematological response.

36. The method of claim 35, wherein the radical scavenger is L-ascorbic acid, L-ascorbic acid 6-palmitate, ubiquinol-10 or α-tocopherol.

37. The method of claim 31, wherein the drug is an antithyroid medication.

38. The method of claim 31, wherein the drug is a sulfonamide.

39. The method of claim 31, wherein the label of the drug comprises a warning that the drug is associated with a risk for neutropenia or agranulocytosis.

40. The method of claim 31, wherein the drug is any of the following compounds or a pharmaceutically acceptable salt thereof: (1) clozapine; (2) quinapril; (3) moexipril; (4) benazepril; (5) enalapril; (6) perindopril erbumine; (7) carbamazepine; (9) lisinopril; (10) trandolapril; (11) ticlopidine; (12) captotril; (13) benazepril; (14) ramipril; (15) penicillamine; (16) propafenone; (17) sulfamethoxazole; (18) zonisamide; (19) leflunomide; (20) sulfacetamide; (21) prednisolone; (22) timolol; (23) dapsone; (24) ofloxacin; (25) levofloxacin; (26) sulfisoxazole; (27) promethazine; (28) amoxicillin; (29) mebendazole; (30) brinzolamide; (31) procainamide and (32) tocainide.

41. The method of claim 31, wherein the drug is any of the following compounds or a pharmaceutically acceptable salt thereof: clozapine, carbamazepine, ticlopidine, procainamide or tocainide.

42. The method of claim 41, wherein the drug is clozapine.

43. The method of claim 42, wherein the individual is diagnosed with a disease selected from the group consisting of: a psychotic disorder, a psychosis secondary to dopaminergic therapy, a psychosis secondary to a coexisting psychiatric disorder in Parkinson's disease, an affective disorder, a personality disorder, a dyskinesia, dementia, mental retardation and polydipsia/hyponatramia.

44 The method of claim 43, wherein the individual is diagnosed with a psychotic disorder.

45. The method of claim 44, wherein the psychotic disorder is schizophrenia, treatment-resistant schizophrenia, psychosis secondary to dopaminergic therapy, or psychosis secondary to coexisting psychiatric disorder in Parkinson's Disease.

46. The method of claim 45, wherein the psychotic disorder is schizophrenia.

47. The method of claim 46, wherein if the HLA-C marker is determined to be absent in the individual, the selected therapy comprises administering to the individual a clozapine drug product which comprises:
(a) clozapine in an amount effective for treating the psychotic disorder; and
(b) prescribing information comprising a statement that the drug product is indicated for treating patients that test negative for the HLA-C marker.

48. The method of claim 47, wherein the prescribing information further comprises a statement that the drug product is indicated for treating the psychotic disorder.

49. The method of claim 1, wherein the adverse herpetological response is agranulocytosis.

50. The method of claim 2, wherein the adverse hematological response is agranulocytosis.

51. The method of claim 3, wherein the adverse hematological response is agranulocytosis.

52. The kit of claim 4, wherein the adverse hematological response is agranulocytosis.

53. The method of claim 31, wherein the adverse hematological response is agranulocytosis.

54. The method of claim 32, wherein the adverse hematological response is agranulocytosis.

55. The method of claim 33, wherein the adverse hematological response is agranulocytosis.

56. The method of claim 34, wherein the adverse hematological response is agranulocytosis.

57. The method of claim 35, wherein the adverse hematological response is agranulocytosis.

58. The method of claim 48, wherein the adverse hematological response is agranulocytosis.
